SEQUENCE LISTING (33 sequences)

SEQ ID NO: 1 - protein, 108 aa, synthetic construct
GARGLPGTAGLPGMKGHRGFPGERGLDGAKGDAGPAGPKGEPGSPGENGAPGQMGPRGPQGPPGPPGPKGNSGEPGAPGSKGDTGAKGEPGPVGVQGPPGPAGEEGKR

SEQ ID NO: 2 - protein, 108 aa, synthetic construct
GFPGERGVQGPPGPAGPRGANGAPGNDGAKGDAGAPGAPGSQGAPGLQGMPGERGAAGLPGPKGDRGDAGPKGADGSPGKDGVRGLTGPIGPPGPAGAPGDKGESGPS

SEQ ID NO: 3 - protein, 138 aa, synthetic construct
GPAGFAGPPGADGQPGAKGEPGDAGAKGDAGPPGPAGPAGPPGPIGESGREGAPGAEGSPGRDGSPGAKGDRGETGPAGPPGFPGERGAPGPAGPAGPVGPVGARGPAGPQGPRGDKGETGEQGDRGIKGHRGFSGLQ

SEQ ID NO: 4 - protein, 111 aa, synthetic construct
GLTGPAGEPGREGSPGADGPPGRDGAAGVKGDRGETGAVGAPGAPGPPGDRGEAGAQGPMGPSGPAGARGIQGPQGPRGDKGEAGEPGERGLKGHRGFTGLQGLPGPPGPS

SEQ ID NO: 5 - protein, 99 aa, synthetic construct
GFPGMKGHRGFDGRNGEKGETGAPGLKGENGLPGENGAPGPMGPRGAPGERGSPGPKGDKGEPGPPGADGVPGKDGPRGPTGPIGPPGPAGQPGDKGEP

SEQ ID NO: 6 - protein, 108 aa, synthetic construct
GFPGMKGHRGFDGRNGEKGETGAPGLKGENGLPGENGAPGPMGPRGAPGERGAKGEPGPRGERGEAGIPGVPGAKGEDGKPGEPGPKGDAGAPGAPGPKGDAGAPGER

SEQ ID NO: 7 - protein, 108 aa, synthetic construct
GFPGMKGHRGFDGRNGEKGETGAPGLKGENGLPGENGAPGPMGPRGAPGERGAKGEPGPRGERGEAGIPGVPGAKGEDGRDGNPGSDGLPGRDGSPGPKGDRGENGSP

SEQ ID NO: 8 - protein, 216 aa, synthetic construct
GARGLPGTAGLPGMKGHRGFPGERGLDGAKGDAGPAGPKGEPGSPGENGAPGQMGPRGPQGPPGPPGPKGNSGEPGAPGSKGDTGAKGEPGPVGVQGPPGPAGEEGKRGFPGERGVQGPPGPAGPRGANGAPGNDGAKGDAGAPGAPGSQGAPGLQGMPGERGAAGLPGPKGDRGDAGPKGADGSPGKDGVRGLTGPIGPPGPAGAPGDKGESGPS

SEQ ID NO: 9 - protein, 216 aa, synthetic construct
GFPGERGVQGPPGPAGPRGANGAPGNDGAKGDAGAPGAPGSQGAPGLQGMPGERGAAGLPGPKGDRGDAGPKGADGSPGKDGVRGLTGPIGPPGPAGAPGDKGESGPSGFPGERGVQGPPGPAGPRGANGAPGNDGAKGDAGAPGAPGSQGAPGLQGMPGERGAAGLPGPKGDRGDAGPKGADGSPGKDGVRGLTGPIGPPGPAGAPGDKGESGPS

SEQ ID NO: 10 - protein, 108 aa, synthetic construct
GPMGPSGPRGLPGPPGAPGPQGFQGPPGEPGEPGASGPMGPRGPPGPPGKNGDDGEAGKPGRPGERGPPGPQGARGLPGTAGLPGMKGHRGFSGLDGAKGDAGPAGPK

SEQ ID NO: 11 - protein, 120 aa, synthetic construct
GPRGLPGPPGAPGPQGFQGPPGEPGEPGASGPMGPRGPPGPPGKNGDDGEAGKPGRPGERGPPGPQGARGLPGTAGLPGMKGPAGSPGFQGLPGPAGPPGEAGKPGEQGVPGDLGAPGPS

SEQ ID NO: 12 - protein, 126 aa, synthetic construct
GANGDPGRPGEPGLPGARGLTGRPGDAGPQGKVGPSGAPGEDGRPGPPGPQGARGQPGVMGFPGPKGANGEPGKAGEKGLPGAPGLRGLPGKDGETGAAGERGSPGAQGLQGPRGLPGTPGTDGPK

SEQ ID NO: 13 - protein, 74 aa, synthetic construct
ADEQEEKAKVRTELIQELAQGLGGIEKKNFPTLGDEDLDHTYMTKLLTYLQEREQAENSWRKRLLKGIQDHALD

SEQ ID NO: 14 - protein, 32 aa, synthetic construct
GEIAAIKQEIAAIKKEIAAIKWEIAAIKQGYG

SEQ ID NO: 15 - protein, 256 aa, synthetic construct
HHHHHHADEQEEKAKVRTELIQELAQGLGGIEKKNFPTLGDEDLDHTYMTKLLTYLQEREQAENSWRKRLLKGIQDHALDLVPRGSPGPPGPPGPPGPPGPPGPPGPPGPPGPPGPPGARGLPGTAGLPGMKGHRGFPGERGLDGAKGDAGPAGPKGEPGSPGENGAPGQMGPRGPQGPPGPPGPKGNSGEPGAPGSKGDTGAKGEPGPVGVQGPPGPAGEEGKRGPPGPPGPPGPPGPPGPPGPPGPPGPPGPPG

SEQ ID NO: 16 - DNA, 771 bp, synthetic construct
  source 1..771: mol_type = other DNA
caccatcaccatcaccacgccgacgagcaagaagaaaaggccaaagttcgcaccgagctgattcaagaactggcgcaaggtctgggcggcatcgaaaagaaaaacttcccgacgctgggcgatgaagatctggaccacacctacatgacgaagctgctgacctatctgcaagaacgtgaacaagccgagaatagctggcgcaaacgtctgctgaaaggcatccaagatcatgcgctggatctggtgccacgtggcagcccgggcccgccgggcccgccgggcccaccgggtccaccgggcccgccgggcccaccgggtccgccgggtccgccgggtccgccgggcccaccgggcgcccgtggtctgccgggcaccgccggtctgccgggcatgaaaggccatcgcggtttcccgggtgaacgtggtctggatggcgccaaaggtgatgcgggtccagccggtccgaaaggcgaaccgggcagcccgggcgaaaatggtgcgccgggccagatgggtccgcgtggtccacaaggcccgccgggcccaccgggcccgaaaggcaatagcggtgaaccgggcgccccgggcagtaaaggcgataccggtgcgaaaggtgaaccgggcccggttggtgttcaaggcccaccgggcccagcgggtgaagaaggtaaacgtggtccgccgggtccaccgggtccaccgggtccaccgggcccaccgggcccgccgggcccaccgggtccgccgggcccgccgggcccaccgggctaa

SEQ ID NO: 17 - protein, 355 aa, synthetic construct
HHHHHHADEQEEKAKVRTELIQELAQGLGGIEKKNFPTLGDEDLDHTYMTKLLTYLQEREQAENSWRKRLLKGIQDHALDLVPRGSPGPPGPPGPPGPPGPPGLTGPAGEPGREGSPGADGPPGRDGAAGVKGDRGETGAVGAPGAPGPPGDRGEAGAQGPMGPSGPAGARGIQGPQGPRGDKGEAGEPGERGLKGHRGFTGLQGLPGPPGPSGPPGPPGPPGPPGLTGPAGEPGREGSPGADGPPGRDGAAGVKGDRGETGAVGAPGAPGPPGDRGEAGAQGPMGPSGPAGARGIQGPQGPRGDKGEAGEPGERGLKGHRGFTGLQGLPGPPGPSGPPGPPGPPGPPGPPGPPG

SEQ ID NO: 18 - DNA, 1068 bp, synthetic construct
  source 1..1068: mol_type = other DNA
catcaccatcaccatcatgcggatgaacaagaagaaaaagcgaaagtgcgcaccgaactgattcaagaactggcgcaaggcctgggcggcattgaaaaaaaaaactttccgaccctgggcgatgaagatctggatcatacctatatgaccaaactgctgacctatctgcaagaacgcgaacaagcggaaaacagctggcgcaaacgcctgctgaaaggcattcaagatcacgccctggacttagtgccgcgcggtagcccgggtccgccgggtccgccgggcccgccgggtccgccgggtccgccgggcttaaccggcccggccggcgaaccgggccgtgagggcagcccgggcgccgatggcccgccgggccgcgacggcgcggccggcgtgaagggcgatcgtggcgaaacgggcgcggtgggtgcgccgggtgcgccgggcccgccgggcgatcgtggtgaagcgggcgcccaaggcccaatgggcccaagtggtccggcgggtgcgcgcggcatccaaggcccgcaaggcccgcgcggtgacaaaggcgaagcgggcgaaccgggcgaacgtggcttaaaaggccaccgcggctttacgggtctgcaaggtttaccgggtccgccgggtccaagtggtccaccgggtccgccgggcccaccgggcccgccgggcttaaccggtccggccggcgagccgggccgtgaaggcagcccgggcgccgatggcccaccgggccgcgatggcgccgcgggcgtgaagggtgatcgcggtgagaccggcgccgtgggcgccccgggcgcgccgggtccgccgggcgaccgcggcgaggccggtgcgcaaggtccgatgggcccgagcggtccggccggtgcgcgtggcattcaaggcccgcaaggcccacgcggtgataaaggcgaagccggtgaaccgggcgaacgcggcctgaaaggccatcgtggttttaccggtttacaaggtctgccgggcccgccgggcccaagtggtccaccgggcccgccgggcccaccgggcccaccgggcccaccgggcccgccgggctaa

SEQ ID NO: 19 - protein, 331 aa, synthetic construct
HHHHHHADEQEEKAKVRTELIQELAQGLGGIEKKNFPTLGDEDLDHTYMTKLLTYLQEREQAENSWRKRLLKGIQDHALDLVPRGSPGPPGPPGPPGPPGPPGFPGMKGHRGFDGRNGEKGETGAPGLKGENGLPGENGAPGPMGPRGAPGERGSPGPKGDKGEPGPPGADGVPGKDGPRGPTGPIGPPGPAGQPGDKGEPGPPGPPGPPGPPGFPGMKGHRGFDGRNGEKGETGAPGLKGENGLPGENGAPGPMGPRGAPGERGSPGPKGDKGEPGPPGADGVPGKDGPRGPTGPIGPPGPAGQPGDKGEPGPPGPPGPPGPPGPPGPPG

SEQ ID NO: 20 - DNA, 996 bp, synthetic construct
  source 1..996: mol_type = other DNA
catcaccatcaccatcatgcggatgaacaagaagaaaaagcgaaagtgcgcaccgaactgattcaagaactggcgcaaggcctgggcggcattgaaaaaaaaaactttccgaccctgggcgatgaagatctggatcatacctatatgaccaaactgctgacctatctgcaagaacgcgaacaagcggaaaacagctggcgcaaacgcctgctgaaaggcattcaagatcatgccctggatttagtgccgcgcggcagcccgggtccaccgggtccgccgggcccgccgggcccaccgggtccgccgggctttccgggcatgaagggccatcgcggttttgatggccgcaacggcgaaaaaggcgaaacgggtgccccgggcctgaaaggcgaaaacggtttaccgggcgagaacggcgcgccgggcccgatgggtccgcgtggtgcgccgggcgaacgcggcagcccgggcccaaaaggtgataagggtgaaccgggtccgccgggcgccgacggtgtgccgggcaaagatggcccgcgcggcccgacgggcccgattggcccgccgggcccggcgggccaaccgggcgacaaaggtgaaccgggcccgccgggcccgccgggcccaccgggtccaccgggttttccgggcatgaagggccatcgcggctttgatggtcgtaacggcgagaagggcgaaaccggtgcgccgggcttaaaaggtgaaaacggcctgccgggcgagaacggcgcgccgggtccgatgggcccacgtggcgccccgggcgagcgcggcagtccgggcccgaagggcgataaaggcgaaccgggcccgccgggcgcggatggcgtgccgggcaaagatggcccacgcggtccaacgggtccgatcggcccgccgggcccggcgggtcagccgggcgataagggtgagccgggcccgccgggcccgccgggcccgccgggcccgccgggcccaccgggcccaccgggttaa

SEQ ID NO: 21 - protein, 7 aa, synthetic construct
LVPRGSP

SEQ ID NO: 22 - protein, 6 aa, synthetic construct
LVPRGS

SEQ ID NO: 23 - protein, 30 aa, synthetic construct
GPPGPPGPPGPPGPPGPPGPPGPPGPPGPP

SEQ ID NO: 24 - protein, 15 aa, synthetic construct
GPPGPPGPPGPPGPP

SEQ ID NO: 25 - protein, 12 aa, synthetic construct
GPPGPPGPPGPP

SEQ ID NO: 26 - protein, 18 aa, synthetic construct
GPPGPPGPPGPPGPPGPP

SEQ ID NO: 27 - protein, 30 aa, synthetic construct
GPPGPPGPKGDPGPPGPPGPKGDPGPPGPP

SEQ ID NO: 28 - protein, 30 aa, synthetic construct
GPPGPKGDPGPPGPKGDPGPPGPKGDPGPP

SEQ ID NO: 29 - protein, 263 aa, synthetic construct
HHHHHHHHGGGGSADEQEEKAKVRTELIQELAQGLGGIEKKNFPTLGDEDLDHTYMTKLLTYLQEREQAENSWRKRLLKGIQDHALDLVPRGSPGPPGPKGDPGPPGPKGDPGPPGPKGDPGPPGFPGERGVQGPPGPAGPRGANGAPGNDGAKGDAGAPGAPGSQGAPGLQGMPGERGAAGLPGPKGDRGDAGPKGADGSPGKDGVRGLTGPIGPPGPAGAPGDKGESGPSGPPGPPGPPGPPGPPGPPGPPGPPGPPGPPG

SEQ ID NO: 30 - protein, 263 aa, synthetic construct
HHHHHHHHGGGGSADEQEEKAKVRTELIQELAQGLGGIEKKNFPTLGDEDLDHTYMTKLLTYLQEREQAENSWRKRLLKGIQDHALDLVPRGSPGPPGPPGPPGPPGPPGPPGPPGPPGPPGPPGFPGERGVQGPPGPAGPRGANGAPGNDGAKGDAGAPGAPGSQGAPGLQGMPGERGAAGLPGPKGDRGDAGPKGADGSPGKDGVRGLTGPIGPPGPAGAPGDKGESGPSGPPGPKGDPGPPGPKGDPGPPGPKGDPGPPG

SEQ ID NO: 31 - protein, 263 aa, synthetic construct
HHHHHHHHGGGGSADEQEEKAKVRTELIQELAQGLGGIEKKNFPTLGDEDLDHTYMTKLLTYLQEREQAENSWRKRLLKGIQDHALDLVPRGSPGPPGPPGPPGPPGPPGPPGPPGPPGPPGPPGFPGERGVQGPPGPAGPRGANGAPGNDGAKGDAGAPGAPGSQGAPGLQGMPGERGAAGLPGPKGDRGDAGPKGADGSPGKDGVRGLTGPIGPPGPAGAPGDKGESGPSGPPGPPGPKGDPGPPGPPGPKGDPGPPGPPG

SEQ ID NO: 32 - protein, 263 aa, synthetic construct
HHHHHHHHGGGGSADEQEEKAKVRTELIQELAQGLGGIEKKNFPTLGDEDLDHTYMTKLLTYLQEREQAENSWRKRLLKGIQDHALDLVPRGSPGPPGPPGPKGDPGPPGPPGPKGDPGPPGPPGFPGERGVQGPPGPAGPRGANGAPGNDGAKGDAGAPGAPGSQGAPGLQGMPGERGAAGLPGPKGDRGDAGPKGADGSPGKDGVRGLTGPIGPPGPAGAPGDKGESGPSGPPGPPGPKGDPGPPGPPGPKGDPGPPGPPG

SEQ ID NO: 33 - protein, 263 aa, synthetic construct
HHHHHHHHGGGGSADEQEEKAKVRTELIQELAQGLGGIEKKNFPTLGDEDLDHTYMTKLLTYLQEREQAENSWRKRLLKGIQDHALDLVPRGSPGPPGPPGPPGPPGPPGPPGPPGPPGPPGPPGFPGERGVQGPPGPAGPRGANGAPGNDGAKGDAGAPGAPGSQGAPGLQGMPGERGAAGLPGPKGDRGDAGPKGADGSPGKDGVRGLTGPIGPPGPAGAPGDKGESGPSGPPGPPGPPGPPGPPGPPGPPGPPGPPGPPG

(19) United States
(12) Patent Application Publication
(10) Pub. No.: US 2026/0152544 A1
(43) Pub. Date: Jun. 4, 2026

(54) COLLAGEN DOMAIN, COLLAGEN, RECOMBINANT COLLAGEN EXPRESSION STRAIN, AND APPLICATIONS THEREOF

(71) Applicant: Jiangnan University, Wuxi (CN)

(72) Inventors: Meng Zhang, Wuxi (CN); Ruixue Zhang, Wuxi (CN); Fei Xu, Wuxi (CN); Jingjing Qi, Wuxi (CN)

(21) Appl. No.: 19/533,671

(22) Filed: Feb. 9, 2026

Related U.S. Application Data
(63) Continuation of application No. PCT/CN2024/116328, filed on Sep. 2, 2024.

(30) Foreign Application Priority Data
Sep. 4, 2023 (CN) 202311135222X
Dec. 31, 2023 (CN) 2023118705631

(51) Int. Cl. C07K 14/78

(52) U.S. Cl. CPC C07K 14/78; C07K 2319/21

(57) ABSTRACT

A collagen domain, collagen, recombinant collagen expression strain, and applications thereof are provided. The present disclosure performs stability prediction and sequence design for human collagen, obtains a collagen structural domain with high homology with natural human collagen, and directly expresses recombinant human collagen with a triple helix structure in Escherichia coli. Each collagen fragment with high thermal stability designed in the present disclosure is properly folded to form a triple helix structure, while collagen fragments with low thermal stability cannot be properly folded. Additionally, designed recombinant human type I collagen with high thermal stability may self-assemble to form periodic alternating light and dark stripes similar to natural type I collagen. The collagen domains and collagens of the present disclosure may be further applied in the biomedicine and tissue engineering fields of biomimetic recombinant collagen with structural functions, and used for tissue culture, dental tissue repair, and the like.

### REFERENCE TO SEQUENCE LISTING

The instant application contains a Sequence Listing in XML format as a file named “PC230007A.xml”, created on 2026 Jan. 27, of 36,320 byte in size, and which is hereby incorporated by reference in its entirety.

### TECHNICAL FIELD

The present disclosure relates to collagen domains, collagens, recombinant collagen expression strain, and applications thereof, and in particular to a method for directly expressing recombinant collagen with a triple helix structure in Escherichia coli, and belongs to the technical field of genetic engineering, where recombinantly expressed human type I collagen may self-assemble into regular biomimetic fibers.

### BACKGROUND

Collagen is the most abundant structural protein in the human body, accounting for about 30% of the total protein in the human body, and is widely distributed in tissues such as bones, tendons, cartilage, and skin. Collagen is composed of three polypeptide chains that intertwine around a central axis to form a right-handed triple helix structure, and the triple helix structure may further assemble into higher-order collagen fibers to exert functions in organisms. Therefore, the triple helix structure of collagen is the basis for its biological functions. TYpes I, II, and III collagens account for 80-90% of the total collagen in the human body, where type I collagen is the most abundant functional protein in animal bodies, the collagen fibers formed by its self-assembly are characterized under transmission electron microscopy, and overlapping and gap regions exhibit a banded morphology (commonly known as a D-period) with alternating light and dark patches. The D-period is considered as a key structural element endowing collagen with various functions, and is related to load-bearing properties of tissues, bone mineralization, and regulation of cell differentiation and adhesion during tissue development. TYpe II collagen exists in the cartilage of ribs, nose, larynx, and trachea, and alleviates the symptoms of joint-related diseases such as osteoarthritis. TYpe III collagen functions together with type I collagen in the skin, ligaments, blood vessels, and joints, and is closely related to the process and quality of skin injury repair. In recent years, with the development of smart biomanufacturing, the demand for high-performance biomimetic biomaterials has increased day by day. Collagen materials have great application potential in skin injury treatment, vascular scaffold engineering, cartilage and bone defect repair, skin care, delivery of hemostatic sponges and drugs (including coatings and medical nanoparticles) due to their good biocompatibility and low immunogenicity.

Currently, collagen is mainly obtained through animal extraction, but its potential immunogenicity limits its application in the field of biomedical materials; and polypeptide chains with collagen characteristic sequences may be obtained through chemical synthesis, but large-scale production of synthesized polypeptide chains is infeasible due to high costs and length limitation. Obtaining recombinant collagen by expressing natural or optimized sequences of human collagen in microorganisms through genetic engineering means has attracted increasing attention and become a research hotspot. This not only solves the viral safety problem existing in traditional extraction methods, but also enables sequence modification according to actual needs to increase the hydrophilicity of collagen and obtain samples with stable quality and high safety.

Microbial expression systems have the advantages of clear genetic background, convenient genetic manipulation, short fermentation cycle, and high expression level, and are widely used for heterologous expression of proteins. However, due to current limitations in achieving human-like post-translational modifications in microbial expression systems, expressed human collagen cannot be modified to fold into a triple helix structure and self-assemble into a higher-order structure. Additionally, due to structural particularity of collagen, the research on the effect of collagen folding is not completely clear currently, resulting in that the sequence design of heterologous expression of human collagen lacks sufficient theoretical support. Therefore, it is still difficult to solve the problem that recombinantly expressed human collagen is folded into a triple helix structure and further assembled into a regular higher-order collagen structure.

Currently, some reports reveal that human collagen may be heterologously expressed in microorganisms such as Escherichia coli, but there exists at least one of the following problems:

(1) With low homology with natural collagen sequences, the sequences are randomly truncated, modified, repeated/spliced based on experience, and have a triple helix structure verified by circular dichroism. For example, a 108-amino-acid collagen domain Col108 reported in the literatures “The self-assembly of a mini-fibril with axial periodicity from a designed collagen-mimetic triple helix” and “To achieve self-assembled collagen mimetic fibrils using designed peptides” is derived from the splicing of four short sequence fragments in a collagen domain of human type I collagen, with only 45.61% homology with a natural sequence; Chinese Patent Application No. CN115521373A discloses a triple helix recombinant human type I collagen, and a preparation method and application thereof, the expressed recombinant human type I collagen has a triple helix structure and may self-assemble into collagen fibers, and the collagen domain fragment of the above patent is the Col108 fragment reported in the above literature with a functional motif inserted, with low homology with the natural sequence; in a thesis titled “Preparation, Structural Characterization and Performance Analysis of Recombinant Human-like Collagen”, a recombinant human-like collagen single fragment of a 38-amino-acid sequence was designed and repeated four or eight times respectively, and expressed in Escherichia coli, and synthesized human-like collagen had a triple helix structure, but any human collagen could not be matched during homology search of a single fragment collagen sequence; Chinese Patent Application No. CN115819557A discloses a triple helix recombinant human type II collagen, and a preparation method and application thereof, expressed recombinant human type II collagen has a triple helix structure and may self-assemble into collagen fibers, and its sequence matches a human collagen sequence for a maximum of seven consecutive amino acid residues; and Chinese Patent Application No. CN115521372A discloses a triple helix recombinant human type III collagen, and a preparation method and application thereof, its sequence matches a natural sequence for a maximum of nine consecutive amino acids, and any human collagen cannot be matched during a sequence homology search.

(2) The expressed collagen has low stability and no triple helix structure at room temperature. For example, in the literature “Recombinant expression of hydroxylated human collagen in Escherichia coli”, folding into a triple helix structure is promoted by co-expressing mimiviral prolyl and lysyl hydroxylases with a human type III collagen fragment, but Tₘ is only 24.3° C., and collagen with low stability easily loses its triple helix structure in in vivo and in vitro applications, thus failing to exert its functions.

(3) The expressed collagen is not characterized by a standardized triple helix structure, and the triple helix structure cannot be determined. According to reports in the “Guidelines for Evaluation of Recombinant Human Collagen Raw Materials”, Nature Protocols, 2006: VOL.1, NO. 6, 2527, and the literature “Selective expression of nonsecreted triple helix and secreted single-chain recombinant collagen fragments in the yeast Pichia pastoris”, a human type III collagen fragment is recombinantly expressed in the yeast Pichia pastoris, and according to the follow-up study literature titled “Expression of recombinant human type I-III collagens in the yeast Pichia pastoris”, prolyl hydroxylase is co-expressed with human types I, II, and III collagens in the yeast Pichia pastoris, but the triple helix structure is not characterized; according to “Production of human type I collagen in yeast reveals unexpected new insights into the molecular assembly of collagen trimers”, folding into a triple helix structure is promoted by co-expressing chicken prolyl hydroxylase with human type I collagen, but Tₘ is measured to be 30° C. only according to a thermal denaturation curve at a wavelength of 197 nm, and an absorption peak at this wavelength is usually similar to a spectrum of unfolded proteins, which cannot be used as a standard method for collagen triple helix characterization, so the triple helix structure cannot be determined; Chinese Patent Application No. CN114276435A discloses a recombinant human type III collagen and an application thereof, a 123-amino-acid sequence segment is selected, a tripeptide sequence in the sequence segment is directionally replaced and repeated, and a specific sequence is connected at a C-terminus, and expressed in the yeast Pichia pastoris without triple helix structure characterization; Chinese Patent Application No. CN114774460A discloses a yeast recombinant human type I triple helix collagen and a preparation method therefor, where a human type I collagen al chain sequence is selected to co-express with hydroxylase, and Chinese Patent Application No. CN114480471A discloses a yeast recombinant human type III triple helix collagen and a preparation method therefor, where a human type III collagen al chain sequence is selected to co-express with hydroxylase; Chinese Patent Application No. CN111087464B discloses a recombinant human type III collagen with a functional structure and an expression method therefor, where a partial sequence fragment of human type III collagen is selected to co-expresses with hydroxylase; Chinese Patent Application No. CN112851797B discloses a recombinant human type III collagen, and a preparation method and use thereof, where fragments of the human type III collagen with the cell-binding ability are spiced and co-expressed with hydroxylase; Chinese Patent Application No. CN116555320A discloses a recombinant human type III triple helix collagen engineering strain, and a construction method and application thereof, where a human type III collagen al chain sequence is selected to co-express with hydroxylase; and Chinese Patent Application No. CN116082494A discloses a recombinant human type III collagen polypeptide, expression vector, expression strain, and a construction method therefor, where a 54-amino-acid polypeptide fragment with strong hydrophilicity and stability is selected from a human type III collagen sequence to express in the yeast Pichia pastoris. The triple helix structure is characterized in none of the above seven patents, and it is unknown whether a triple helix structure may be really formed.

Additionally, among preliminary research findings of the inventor's team, Chinese Patent Application No. CN111333715B (a preparation method for type I-like collagen fibers) discloses a collagen sequence based on (GPP), sequences at the N-terminus and C-terminus, where Gly-Xaa-Yaa triplets are inserted consecutively between the termini to form banded fibers with periodic alternating light and dark stripes, and Chinese Patent Application No. CN111499729B (a method for regulating a stripe period length of type I-like collagen fibers), based on (GPP), sequences at the N-terminus and C-terminus, collagen sequences with different numbers of Gly-Xaa-Yaa triplets are inserted consecutively between the termini to form banded fibers with periodic alternating light and dark stripes of different dark stripe lengths, and human collagen sequences are systematically designed in none of the patents; and according to the master's thesis of Yan Haojie from the inventor's team that is titled “Hierarchical Self-Assembly of Collagen Polypeptides Induced by Multiple Non-Covalent Interactions”, a human type I collagen sequence fragment is selected and expressed in Escherichia coli to form a triple helix structure, but a fiber structure similar to natural human collagen is not formed by assembling.

Therefore, it is necessary to develop a collagen sequence with high homology with natural human collagen and capable of achieving exogenous expression of a triple helix structure based on systematic thermal stability analysis

### SUMMARY

In order to solve at least one of the above problems existing in recombinant human collagen, such as low homology with natural human collagen, difficulty in heterologous expression to form a triple helix structure, or difficulty in further self-assembly to form a higher-order structure, the present disclosure obtains a collagen domain (also known as a collagen structural domain or a collagen domain) with high homology with natural collagen by truncating collagen fragments of human types I, II, and III collagens for sequence splicing and design through systematic thermal stability prediction and analysis; and further, repeating sequence modules are introduced at both termini of the collagen domain, and when a designed collagen sequence is expressed in Escherichia coli, it is found that each designed collagen fragment with high thermal stability may be properly folded to form a triple helix structure, while collagen fragments with low thermal stability cannot be properly folded. Additionally, designed recombinant human type I collagen with high thermal stability may self-assemble to form periodic alternating light and dark stripes similar to natural type I collagen. The present disclosure develops sequences of collagen with high homology with natural human collagen and capable of achieving exogenous expression of a triple helix structure, and achieves expression thereof, thus meeting the demand for recombinant collagen with structural functions in the fields of biomedicine and tissue engineering.

A first objective of the present disclosure is to provide an amino acid sequence encoding a collagen domain, and the amino acid sequence includes:
(1) an amino acid sequence as shown in any one of SEQ ID NO: 1-7; or
(2) an amino acid sequence obtained by combining any two sequences as shown in SEQ ID NO: 1-3; or
(3) an amino acid sequence obtained by repeating a sequence as shown in any one of SEQ ID NO: 1-7 for 2-3 times.

In an embodiment, the amino acid sequences shown in SEQ ID NO:1-7 are obtained by truncating collagen fragments of natural human types I, II, and III collagens or further performing sequence splicing and design.

In an embodiment, the amino acid sequences shown in SEQ ID NO:1-7 are obtained by performing thermal stability prediction of natural human types I, II, and III collagens, and selecting sequences with high predicted Tₘ values for truncation or splicing. A predicted Tₘ value of a collagen triple helix structure with the amino acid sequence as a collagen domain is 38-39° C.

The predicted Tₘ value of a collagen triple helix structure with the amino acid sequence as a collagen domain is specifically predicted by the following method: taking a first triplet unit (XYG) of the triple helix structure as a starting point of continuous numbering, calculating average relative stability for each XYG triplet to obtain a thermal stability value of each triplet; then taking n consecutive triplets, calculating a mean of thermal stability values of these n consecutive triplets, to obtain a predicted thermal stability value of a collagen domain sequence; where a thermal stability value of a single triplet i refers to a thermal stability value of a window composed of 10 consecutive triplets in an interval [i−5, i+5]; and a thermal stability value T_{windows} of the window is determined by a window main-chain propensity value T_{bb} and a window side-chain interaction value T_{side},

$${T}_{windows} = \frac{{T}_{bb}}{60} + \frac{{T}_{side}}{10} .$$

A second objective of the present disclosure is to provide a single protein chain for expressing collagen, and the single protein chain contains the above amino acid sequence encoding a collagen domain.

In an embodiment, a structure of the single protein chain includes: a folding domain, a repeating sequence module, and a collagen domain.

In an embodiment, the structure of the single protein chain from the N-terminus to the C-terminus includes: a folding domain, {a repeating sequence module, a collagen domain}m, and a repeating sequence module, where m is an integer and m≥1.

Optionally, m is 1 or 2.

In an embodiment, a glycine may be further added at a terminus of an amino acid sequence of a single collagen chain.

In an embodiment, introduction of the folding domain assists collagen folding to form a triple helix structure.

Optionally, V-domain is a folding domain, and an amino acid sequence thereof is shown in SEQ ID NO:13; and optionally, coiled-coil domain is a folding domain, and an amino acid sequence thereof is shown in SEQ ID NO:14.

In an embodiment, introduction of the repeating sequence module assists folding of a collagen triple helix and improves its thermal stability. Optionally, a plurality of repeating sequence modules may be arranged and located at both termini of a collagen domain or both termini of a plurality of collagen domains; and for example, when type II collagen is expressed, a plurality of collagen domains may be arranged, and the plurality of collagen domains are connected through repeating sequence modules. Optionally, sequences of the repeating sequence modules may be identical or different.

In an embodiment, the repeating sequence modules employ (GPP)ₙ. Optionally, when a plurality of repeating sequence modules are arranged, values of n in each repeating sequence module (GPP)ₙ may be identical or different. Optionally, by adjusting a value of n in the repeating sequence module (GPP)ₙ, molecules may be further assembled to form a fiber structure. Optionally, in a (GPP)ₙ Collagen (GPP)ₙ pattern that may be assembled into a fibrous morphology, two n values are equal, a value of n in (GPP)ₙ satisfies 5<n≤30, and a value of n disclosed in Chinese Patent Application No. CN 111333715 B disclosed in the preliminary research by the inventor's team may be referred to. Optionally, for a (GPP)ₙ Collagen (GPP)ₙ Collagen (GPP)ₙ pattern of forming a triple helix for types II and III collagens, three n values may be unequal.

In an embodiment, the folding domain and the repeating sequence module are linked through an enzyme cleavage site, such as LVPRGSP (a sequence as shown in SEQ ID NO:21). Optionally, the folding domain V-domain and the repeating sequence module (GPP)ₙ are linked through LVPRGS (a sequence as shown in SEQ ID NO:22).

In an embodiment, a structure of the single protein chain for expressing collagen, from the N-terminus to the C-terminus, sequentially includes: a folding domain, an enzyme cleavage site, {a repeating sequence module, a collagen domain}m, and a repeating sequence module; where m is greater than or equal to 1. Optionally, m is 1 or 2.

In an embodiment, a front terminus (the N-terminus) of the folding domain has a 6×His tag.

In an embodiment, the structure of the single protein chain for expressing collagen is shown in FIG. 10; or the structure is shown in FIG. 11.

In an embodiment, the structure of the single protein chain for expressing collagen is as follows: a first repeating sequence module-a collagen domain-a second repeating sequence module; at least one of the first repeating sequence module and the second repeating sequence module has an amino acid sequence as shown in SEQ ID NO:27 (abbreviated as KD2) or SEQ ID NO:28 (abbreviated as KD3), and the other module has an amino acid sequence as shown in SEQ ID NO: 23, i.e., (GPP)₁₀ (abbreviated as P10);

or both the first repeating sequence module and the second repeating sequence module have the amino acid sequence as shown in SEQ ID NO:23.

Optionally, an amino acid sequence of the first repeating sequence module of the single collagen chain is as shown in SEQ ID NO:23, and an amino acid sequence of the second repeating sequence module is as shown in SEQ ID NO:27;
optionally, an amino acid sequence of the first repeating sequence module of the single collagen chain is as shown in SEQ ID NO:28, and an amino acid sequence of the second repeating sequence module is as shown in SEQ ID NO:23;
optionally, an amino acid sequence of the first repeating sequence module of the single collagen chain is as shown in SEQ ID NO:23, and an amino acid sequence of the second repeating sequence module is as shown in SEQ ID NO:28;
optionally, both the first repeating sequence module and the second repeating sequence module of the single collagen chain have an amino acid sequence as shown in SEQ ID NO:27; and
optionally, both the first repeating sequence module and the second repeating sequence module of the single collagen chain have an amino acid sequence as shown in SEQ ID NO:23.

In an embodiment, a structure of the single protein chain of the collagen from the N-terminus to the C-terminus includes: {KD2, a collagen domain}m, KD2; or {P10, a collagen domain}m, KD2; or {KD3, a collagen domain}m, P10; or {P10, a collagen domain}m, KD3; or {P10, a collagen domain}m, P10; where m=1.

A third objective of the present disclosure is to provide a nucleotide sequence encoding the collagen domain, or a nucleotide sequence encoding the single protein chain for expressing collagen, or a gene encoding the single protein chain for expressing collagen, and a plasmid or cell expressing the gene.

Optionally, the plasmid may be a plasmid of pColdIII series or pET series.

Optionally, the cell is an Escherichia coli cell, including E. coli BL21, E. coli BL21 (DE3), E. coli Rosetta (DE3), E. coli BL21 (DE3) pLysS/pLysE, or E. coli Origami2 (DE3).

Optionally, the cell is E. coli BL21 (DE3).

A fourth objective of the present disclosure is to provide a collagen, and the collagen is composed of three single protein chains that intertwine around a common central axis to form a triple helix structure.

In an embodiment, the structure of the single protein chain of the collagen from the N-terminus to the C-terminus includes: {a repeating sequence module, a collagen domain}m, and a repeating sequence module, where m is an integer and m≥1.

In an embodiment, both the repeating sequence module and the repeating sequence module employ (GPP)ₙ; and optionally, a value of n in (GPP)ₙ satisfies 5<n≤30.

In an embodiment, a structure of the single protein chain for expressing collagen is as follows: a first repeating sequence module-a collagen domain-a second repeating sequence module; at least one of the first repeating sequence module and the second repeating sequence module has an amino acid sequence as shown in SEQ ID NO:27 (abbreviated as KD2) or SEQ ID NO:28 (abbreviated as KD3), and the other module has an amino acid sequence as shown in SEQ ID NO: 23, i.e., (GPP)₁₀ (abbreviated as P10);

or both the first repeating sequence module and the second repeating sequence module have the amino acid sequence as shown in SEQ ID NO:23.

In an embodiment, a structure of the single protein chain of the collagen from the N-terminus to the C-terminus includes: {KD2, a collagen domain}m, KD2; or {P10, a collagen domain}m, KD2; or {KD3, a collagen domain}m, P10; or {P10, a collagen domain}m, KD3; or {P10, a collagen domain}m, P10; where m=1.

A fifth objective of the present disclosure is to provide collagen fibers formed by high-polymerization-induced self-assembly of the collagen.

In an embodiment, the collagen is type I collagen. Optionally, the collagen fibers are fibers with periodic alternating light and dark stripes; and optionally, the collagen fibers exhibit a light stripe morphology under negative-stain transmission electron microscopy (TEM).

In an embodiment, the collagen fibers may be obtained by adjusting a value of n in (GPP)ₙ; and optionally, a repeating sequence module is modified to (GPP)₁₀, and a corresponding light stripe length is 10 nm.

In an embodiment, an amino acid sequence of the collagen domain of the present disclosure is introduced into a collagen domain region of type I collagen, such that dark stripes in collagen fibers satisfy: (number of amino acids in the collagen domain region÷3×0.9)±1 nm.

A sixth objective of the present disclosure is to provide a product containing the collagen of the present disclosure, where the collagen is a collagen containing a triple helix structure; the collagen with the triple helix structure has the collagen domain of the present disclosure, or is formed by the single protein chains of the present disclosure that intertwine around a common central axis.

The product is a product in the fields of beauty, chemical engineering, food health, medical treatment/biomedicine, cosmetics, and feed, such as beauty cosmetics (a facial mask, an essence, a cream, or the like), an artificial collagen casing, a nutritional health product (a collagen powder, an oral liquid), a medical dressing, a hemostatic material, an artificial bone scaffold, an injectable filler, an artificial blood vessel, an eye drop, a sustained-release drug carrier, or the like.

In an embodiment, the product is a dressing, including a collagen membrane layer containing the triple helix structure of the present disclosure; optionally, a drug-loaded cellulose membrane layer is further included; and optionally, the number of the collagen membrane layers is 2-4, the number of the drug-loaded cellulose membrane layers is 1-3, and one drug-loaded cellulose membrane layer is correspondingly disposed between two collagen membrane layers.

In an embodiment, the product is an injectable filler, and is prepared by the following steps: dissolving the collagen of the present disclosure in water for injection to form a collagen solution with a mass fraction of 1.0%-5.0%, dissolving medical-grade sodium hyaluronate in water for injection to form a medical-grade sodium hyaluronate solution with a mass fraction of 0.5%-2.0%, mixing the two solutions in a certain ratio, then adding a certain amount of medical-grade hydroxyapatite, mixing and stirring, adding an appropriate amount of medical-grade glycerol during stirring, and performing vacuum degassing after mixing uniformly, to obtain a finished product.

In an embodiment, the product is an artificial bone scaffold material, which is prepared by the following steps: configuring the collagen of the present disclosure into a collagen solution with a certain concentration, then adjusting a pH value to 6.5-7.5, adding transglutaminase and nano-hydroxyapatite, transferring a resulting mixture to a mold for reaction for a period of time, cooling, inactivating the enzyme, and freeze-drying to obtain the artificial bone scaffold material.

In an embodiment, the product contains the above single protein chain or the above collagen or the above collagen fibers.

In an embodiment, the product further contains one or more of a vitamin, a mineral, hyaluronic acid, a natural polysaccharide, an essential oil, a polyphenolic substance, and a natural plant extract.

A seventh objective of the present disclosure is to provide an application in preparing collagen-containing products in the fields of biology, chemical engineering, food, medicine, biomaterials, tissue engineering, or cosmetics, and the application includes using an amino acid sequence encoding the collagen domain, the single protein chain, the collagen, the collagen fibers of the present disclosure, or a nucleotide sequence encoding the collagen domain, a nucleotide sequence encoding the single protein chain for expressing collagen, a gene encoding the single protein chain for expressing collagen, or a plasmid or cell expressing the gene.

The present disclosure further provides a method for expressing collagen with a triple helix structure, and the method is used to construct collagen with the collagen domain of the present disclosure having a specific amino acid sequence; where an amino acid sequence encoding the collagen domain includes:
(1) an amino acid sequence as shown in any one of SEQ ID NO:1-7; or
(2) an amino acid sequence obtained by combining any two sequences as shown in SEQ ID NO: 1-3; or
(3) an amino acid sequence obtained by repeating a sequence as shown in any one of SEQ ID NO: 1-7 for 2-3 times.

In an embodiment, the method includes: constructing a single protein chain for expressing collagen, and expressing in a host to obtain recombinant collagen with a triple helix structure, where the structure of the single protein chain for expressing collagen, from the N-terminus to the C-terminus, sequentially includes: a folding domain, an enzyme cleavage site, {a repeating sequence module, a collagen domain}m, and a repeating sequence module; where m is greater than or equal to 1. Optionally, m is 1 or 2.

In an embodiment, the method further includes: performing enzymic digestion to remove a folding domain of the recombinant collagen to obtain collagen that still maintains a triple helix structure.

The present disclosure further provides a method for improving stability, solubility, or yield of collagen, and the method includes: linking a first repeating sequence module to the N-terminus of a collagen domain, and linking a second repeating sequence module to the C-terminus to construct a single collagen chain with a structure of first repeating sequence module-collagen domain-second repeating sequence module, and expressing through a host cell; and

at least one of the first repeating sequence module and the second repeating sequence module has an amino acid sequence as shown in SEQ ID NO:27 (abbreviated as KD2) or SEQ ID NO: 28 (abbreviated as KD3), and the other module has an amino acid sequence as shown in SEQ ID NO: 23, i.e., (GPP)₁₀ (abbreviated as P10).

In an embodiment, an amino acid sequence of the collagen domain includes:
(1) an amino acid sequence as shown in any one of SEQ ID NO: 1-7; or
(2) an amino acid sequence obtained by combining any two sequences as shown in SEQ ID NO: 1-3; or
(3) an amino acid sequence obtained by repeating a sequence as shown in any one of SEQ ID NO: 1-7 for 2-3 times.

The recombinant human collagen provided by the present disclosure may be folded to form a triple helix structure and controllably self-assemble into a regular higher-order biomimetic fiber structure. The present disclosure selects sequences with high/low predicted Tₘ values by performing thermal stability prediction of natural types I, II, and III collagens to form collagen domains, and optionally introduces designed different types of collagen sequences into a collagen domain with a structure shown in FIG. 10 or FIG. 11; introduction of the folding domain (such as V-domain with a sequence as shown in SEQ ID NO: 13 or coiled-coil domain with a sequence as shown in SEQ ID NO:14) assists collagen folding to form a triple helix structure; introduction of the repeating sequence module assists folding of a collagen triple helix and improves its thermal stability; and by adjusting a value of n in (GPP)ₙ, molecules may be further assembled to form a fiber structure, which exhibits a light stripe morphology under negative-stain transmission electron microscopy (TEM).

The present disclosure further performs thermal stability analysis and TEM characterization on the obtained recombinant collagen to determine high thermal stability fragments in the designed types I, II, and III collagens. Although an actual Tₘ value deviates from a predicted Tₘ value (38-39° C.), proper folding to form a triple helix structure may be achieved, while predicted low thermal stability fragments cannot be folded to form a triple helix structure.

### BENEFICIAL EFFECTS

1. The collagen structural domain (collagen domain) of the present disclosure is obtained by truncating collagen fragments of natural human types I, II, and III collagens for sequence splicing and design, and has high homology with natural human collagen; and fragments directly truncated from natural human collagen have 100% homology with those of natural sequences, and a collagen domain sequence obtained by splicing has more than 57% homology with a natural sequence.
2. The present disclosure successfully achieves the heterologous expression of high thermal stability fragments of different types of human collagens in Escherichia coli by performing sequence screening and design based on the thermal stability prediction of human collagen sequences.
3. A predicted Tₘ of a human collagen sequence of the present disclosure is 38-39° C.; and a thermal denaturation temperature Tₘ of a collagen domain measured by circular dichroism is further relatively close to a human body temperature.
4. The present disclosure, by using sequences with high homology with those of human collagen, achieves the expression of human type I collagen with a triple helix structure and capable of self-assembling to form a regular higher-order biomimetic fiber structure in Escherichia coli, and resolves the current dilemma of recombinant human collagen expression. Moreover, all prepared human type I collagens are capable of self-assembling into fibers with periodic alternating light and dark stripes, and are similar to type I collagen in the morphology, which meet the demand for recombinant collagen with structural functions in the fields of biomedicine and tissue engineering.
5. An integrin binding site is introduced/incorporated into a high-stability human type I collagen sequence designed in the present disclosure, and the collagen sequence is folded to form a stable triple helix structure and self-assembles into a fibrous morphology. The present disclosure provides a reference for applying recombinant collagen in tissue culture, dental tissue repair, and other fields, and for further introducing any other functional motif into a collagen sequence.
6. By modifying a repeating sequence (GPP)₁₀, the present disclosure obtains a plurality of collagens composed of different repeating sequences, and compared with the collagen (V-P10BP10) composed of (GPP)₁₀, modified collagens (V-KD3BP10, V-P10BKD2, V-KD2BKD2, V-P10BKD3) feature a yield increase by more than 23.4%, an increase in heat resistance temperature by 2° C., and enhanced solubility, and good salt resistance.

### BRIEF DESCRIPTION OF THE DRAWINGS

FIG. 1 is a schematic diagram of axial and lateral side-chain interactions in a triple helix structure of collagen.

FIG. 2 is a relative stability chart of type I collagen.

FIG. 3 is a schematic diagram of collagen sequence design.

FIG. 4 is a chart of SDS-PAGE identification after collagen purification, where lanes 1-7 are purified V-HC1-1, V-HC1-2, V-HC1-3, V-HC1-12, V-HC1-22, V-HC1F, and V-HC1E respectively, an arrow represents a target band, and M: protein marker.

FIG. 5 is a chart of SDS-PAGE identification after collagen purification, where lanes 1-2 and 4-6 are purified V-HC2A, V-HC2B, V-HC3A, V-HC3B, and V-HC3C respectively, and an arrow represents a target band, and M: protein marker.

FIG. 6A illustrates full-wavelength spectra of designed type I collagen.

FIG. 6B illustrates thermal denaturation curves of designed type I collagen.

FIG. 7A illustrates a full-wavelength spectrum and a thermal denaturation curve of designed type II collagen.

FIG. 7B illustrates a full-wavelength spectrum and a thermal denaturation curve of designed type III collagen.

FIG. 8A illustrates TEM characterization of self-assembled fibers HC1-1, HC1-2, and HC1-3 of designed collagen.

FIG. 8B is a statistical diagram of band widths of light and dark stripes of self-assembled fibers HC1-1, HC1-2, and HC1-3 of designed collagen.

FIG. 9A illustrates TEM characterization of self-assembled fibers HC1-12 and HC1-22 of designed collagen.

FIG. 9B is a statistical diagram of band widths of light and dark stripes of self-assembled fibers HC1-12 and HC1-22 of designed collagen.

FIG. 10 illustrates a single protein chain for expressing collagen, with a structure of folding domain-repeating sequence-collagen domain-repeating sequence.

FIG. 11 illustrates a single protein chain for expressing collagen, with a structure of folding domain-repeating sequence-collagen domain-repeating sequence-collagen domain-repeating sequence.

FIG. 12 is an SDS-PAGE diagram of collagen with V-domain after trypsin digestion, where numbers 1-5 correspond to sequences P10BP10, KD3BP10, P10BKD2, KD2BKD2, and P10BKD3 respectively, an arrow represents a target band, and M: protein marker.

FIG. 13 is a yield diagram of collagens with different sequences.

FIG. 14 illustrates a full-wavelength spectrum, a thermal denaturation curve, and a first derivative curve of the thermal denaturation curve of collagen.

FIG. 15A illustrates a full-wavelength spectrum, a thermal denaturation curve, and a first derivative curve of the thermal denaturation curve of collagen KD3BP10 at 0 mM, 10 mM, and 100 mM.

FIG. 15B illustrates a full-wavelength spectrum, a thermal denaturation curve, and a first derivative curve of the thermal denaturation curve of collagen P10BKD2 at 0 mM, 10 mM, and 100 mM.

FIG. 15C illustrates a full-wavelength spectrum, a thermal denaturation curve, and a first derivative curve of the thermal denaturation curve of collagen KD2BKD2 at 0 mM, 10 mM, and 100 mM.

FIG. 15D illustrates a full-wavelength spectrum, a thermal denaturation curve, and a first derivative curve of the thermal denaturation curve of collagen P10BKD3 at 0 mM, 10 mM, and 100 mM.

FIG. 16 illustrates photos of samples of different collagen samples with concentrations of 2 mmol/L dissolved in 10 mM sodium phosphate buffers and placed at 4° C. for 1 day.

### DETAILED DESCRIPTIONS OF THE EMBODIMENTS

### Culture Media:

An LB medium (g/L): tryptone (10), a yeast extract (5), NaCl (10), agar powder (15) (a solid medium);
a TB medium (g/L): tryptone (12), a yeast extract (24), glycerol (4 mL), KH₂PO₄ (2.31), K₂HPO₄ (12.54);

Culture method: aspirating 50 μL of a bacterial solution from a glycerol tube containing a target gene to 20 mL of a LB (Amp-resistant) medium, and culturing overnight at 37° C. with shaking at 200 r/min. Transferring 1% (v/v) of the overnight culture to 100 mL of a TB fermentation broth (Amp-resistant), culturing at 37° C. with shaking at 200 r/min for 24 h, adding IPTG to a final concentration of 1 mmol/L, fermenting and culturing at 25° C. with shaking at 200 r/min for 10 h, and then for fermenting at 15° C. for 14 h.

Protein purification method: After fermentation, collecting bacterial cells, breaking and centrifuging the bacterial cells to collect a supernatant, and filtering through a 0.45 μm aqueous filter membrane. Then performing affinity purification with 5 mL of His Trap™ HP, first equilibrating with 5 column volumes of a binding buffer A (20 mmol/L Na₂HPO₄, 20 mmol/L NaH₂PO₄, 500 mmol/L NaCl, 10 mmol/L Iminazole, pH 7.4), and then loading samples at a flow rate of 5 mL/min. After loading, performing gradient elution with an elution buffer B (20 mmol/L Na₂HPO₄, 20 mmol/L NaH₂PO₄, 500 mmol/L NaCl, 500 mmol/L Iminazole, and pH 7.4) to obtain a target protein, and analyzing purification conditions via SDS-PAGE.

Trypsin digestion and desalting: dissolving the purified collagen in water to a concentration of 4 mg/mL, taking 200 μL of samples respectively, adding trypsin with a concentration of 2.5 g/L according to molar ratios of 20:1, 200:1, and 2000:1, digesting in a water bath at 16° C., sampling every 3 h, finally digesting in a constant temperature incubator for 12 h, and verifying the purity by SDS-PAGE analysis. After digestion under optimal conditions, desalting with HiTrap Desalting, collecting a peak sample, and performing vacuum freeze-drying.

Sample stability identification: performing vacuum freeze-drying of a desalted sample, and identifying a full wavelength and thermal stability thereof by circular dichroism. Specific steps include: dissolving the freeze-dried sample in a 10 mmol/L sodium phosphate buffer with pH 7.0 to form a solution of 1 mg/mL, equilibrating at 4° C. for 48 h, and then performing circular dichroism identification. Data of full wavelength is acquired by measuring CD spectrum of 190-250 nm at 4° C. with an interval of 1 nm, and average scanning time of 5 s. A thermal denaturation curve is obtained by monitoring CD signals at 225 nm and raising the temperature from 4° C. to 70° C. at a heating rate of 10° C./h, while equilibrating for 8 s at each temperature, and a melting temperature (Tₘ) is obtained by taking a median of absorbance values corresponding to a fitted thermal denaturation curve at 4° C. and 70° C., which represents the sample stability.

Transmission electron microscopy (TEM) characterization: dissolving a freeze-dried collagen sample in a 10 mmol/L sodium phosphate buffer with pH 7.0 to prepare a solution with a final concentration of 0.5 mmol/L, and self-assembling at 4° C. for 4 days. Applying 5 μL of the assembled sample onto a copper grid for adsorption for 30 s, absorbing excess liquid with filter paper, then adding 5 μL of 0.75% phosphotungstic acid for negative staining, absorbing a staining solution after 20 s, air-drying, and observing imaging results using a Hitachi H-7650 transmission electron microscope at a voltage of 80 kV. Selecting at least 5 clear TEM images, measuring band widths of light and dark stripes with ImageJ, and measuring each sample at least 200 times to calculate an average value.

### Thermal Stability Analysis Method:

The steps include:
(1) expressing a gene encoding a protein with a structure as shown in FIG. 10 or FIG. 11 in Escherichia coli BL21 (DE3);
(2) purifying an intracellularly expressed product to obtain a purified protein, and performing SDS-PAGE identification;
(3) digesting a sample purified with trypsin, and performing desalting and freeze-drying after SDS-PAGE identification that V-domain is completely removed;
(4) dissolving a freeze-dried collagen sample in a 10 mmol/L sodium phosphate buffer to form a solution with a final concentration of 1 mg/mL, equilibrating at 4° C. for 48 h, and performing circular dichroism identification through full-wavelength scanning and thermal denaturation temperature scanning; and
(5) dissolving a freeze-dried type I collagen sample in a 10 mmol/L sodium phosphate buffer to form a collagen solution with a final concentration of 0.5 mmol/L, equilibrating at 4° C. for 4 days, and then performing TEM characterization.

### Example 1: Collagen Domain Sequence Design

Protein calculation analysis and thermal stability prediction were performed on a full-length sequence of natural human collagen to obtain sequence fragments with high thermal stability, and the fragments were directly truncated or the truncated fragments were further spliced to obtain a collagen domain sequence. A target collagen domain sequence was obtained, and a predicted thermal stability Tₘ of a collagen triple helix structure with this sequence as a collagen domain was 38-39° C.

The predicted thermal stability (Tₘ) of a collagen triple helix structure formed by the collagen domain sequence is specifically predicted by the following method: taking a first triplet unit (XYG) of the triple helix structure as a starting point of continuous numbering, average relative stability for each XYG triplet was calculated to obtain a thermal stability value of each triplet; then taking n consecutive triplets, a mean of thermal stability values of these n consecutive triplets was calculated, to obtain a predicted thermal stability value of a collagen domain sequence. The target collagen domain sequence of the present disclosure ensures that the predicted thermal stability Tₘ of the collagen domain sequence is 38-39° C. while maximizing n as much as possible.

A thermal stability value of a single triplet i refers to a thermal stability value of a window composed of 10 consecutive triplets in an interval [i−5, i+5].

A thermal stability value T_{windows} of the window is determined by a window main-chain propensity value T_{bb} and a window side-chain interaction value T_{side},

$${T}_{windows} = \frac{{T}_{b b}}{60} + \frac{{T}_{side}}{10} .$$

The T_{bb} is obtained by the following method:
(1) Based on a host-guest system, where a most stable triplet Pro-Hyp-Gly served as the host, guests were constructed by only performing single-point mutations of an X-position Pro to 19 non-Pro residues, and a thermal stability value Tₘ of each guest was determined, to obtain main-chain propensity values of different X positions; and similarly, guests were constructed by only performing single-point mutations of a Y-position Hyp in a Pro-Hyp-Gly triplet to 20 natural amino acids, and a thermal stability value Tₘ of each guest was determined to obtain main-chain propensity values of different Y positions; and
(2) a main-chain propensity value of any triplet XYG was calculated based on the corresponding main-chain propensity value of the X position and the main-chain propensity value of the Y position in the step (1) according to types of residues at the X position and the Y position in the triplet, that is, the corresponding main-chain propensity value T_{X} of the X position and the main-chain propensity value T_{Y} of the Y position are summed, i.e., T_{X}+T_{Y}. For example, a main-chain propensity value of an Ala-Ala-Gly triplet is T_{X}+T_{Y}, T_{X} (X=Ala) represents a measured Tₘ value of Ala-Hyp-Gly, and T_{Y} (Y=Ala) represents a measured Tₘ value of Pro-Ala-Gly; and
(3) a window main-chain propensity value T_{bb} was obtained by summing main-chain propensity values of all triplets in the window according to a method for calculating the main-chain propensity value of any triplet XYG in the step (2), where the window includes three chains, and each chain has 10 triplets (i.e., including 60 triplets), that is,

$${T}_{bb} = \sum ( {T}_{X} + {T}_{Y} ) .$$

The T_{side} is derived from all side-chain interactions in the window,

$${T}_{side} = \sum Δ {T}_{Lat} + \sum Δ {T}_{Axi} .$$

ΔT_{Axi} represents an axial interaction value between adjacent triplets across two chains, and ΔT_{Lat} represents a lateral interaction value between adjacent triplets across two chains.

A triple helix folding structure constrains interactions between adjacent chains into two types of geometric structures: axial and lateral (FIG. 1). An interaction between the Y position and the X position of two adjacent chains, where a direction of the interaction is parallel to a helical axis, is called an axial interaction; and an interaction between the Y position and the X position of two adjacent chains, where a direction of the interaction is perpendicular to the helical axis, is called a lateral interaction.

ΔT_{Axi} and ΔT_{Lat} are measured and calculated through double mutation experiments, respectively representing differences between thermal stability of double mutations at the Y position and the X position of the axial or lateral geometric structure and a sum of stabilities of single mutations at the Y position or the X position, as shown in the following formula:

$$Δ T = {T}_{YX} - ( {T}_{YP} + {T}_{OX} ) - {T}_{OP} ;$$

T_{OP} represents a Tₘ value experimentally measured when Hyp is at the Y position and Pro is the X position; T_{OX} represents a Tₘ value experimentally measured when the X position is single-point mutated and Hyp is still at the Y position; T_{YP} represents a Tₘ value experimentally measured when the Y position is single-point mutated and Pro is still at the X position; and T_{YX} represents a Tₘ value experimentally measured when both the Y position and the X position are double-mutated, that is, Hyp is not at the Y position, and Pro is not at the X position.

For example, when a lateral interaction value (ΔT_{Lat}) is calculated when Lys is at the Y position and Asp is at the X position, a thermal stability value determined based on double mutations is T_{YX} (Y=Lys, X=Asp), a corresponding Tₘ value determined based on single-point mutation at the X position is T_{OX} (X=Asp, Y=Hyp), and a Tₘ value determined based on single-point mutation at the Y position is T_{YP} (Y=Lys, X=Pro). A thermal stability value T_{OP} of the host remains unchanged, indicating that Y=Hyp, X=Pro. The Y position of a lateral interaction may mutate into other 20 natural amino acids, the X position may mutate into other 19 natural amino acids, 20×19=380 combinations are formed based on different Y and X pairs, corresponding to 380 lateral interaction values (ΔT_{Lat}). Axial interaction values (ΔT_{Axi}) are determined by a similar method, 380 axial interaction values may further be obtained, and for details, refer to the master's thesis of Liu Han from the inventor's team that titles “Effects of Amino Acid Composition on Thermal Stability of Collagen-like Polypeptides”.

A window unit contains three chains (a, b, and c chains) arranged staggered by one residue, and each chain has 10 triplets (as shown in FIG. 1). 10 lateral interaction pairs and nine axial interaction pairs are contained between the chain a and the chain b and between the chain b and the chain c, respectively; and nine lateral interaction pairs and eight axial interaction pairs are contained between the chain c and the chain a. Therefore, in a window composed of 10 triplets, 29 lateral interaction values and 26 axial interaction pairs exist among three chains, and they are summed to obtain ΣΔT_{Lat} and ΣΔT_{Axi}. A total contribution of all axial and lateral side-chain interaction pairs in the window is T_{side}.

In the above method, when Tₘ is experimentally determined, thermal stability measurement is performed by circular dichroism, specifically including: A freeze-dried pure host or guest collagen peptide powder was weighed and dissolved in a 10 mM phosphate buffer (pH 7.0) to prepare a high-concentration (1 mM) stock solution. Stock solutions of a host peptide and a guest peptide were further diluted to a final concentration of 0.2 mM, mixed in a ratio of 1:1:1 between the chain a, the chain b, and the chain c, and heated at 80° C. for 10 minutes to unfold the folded triple helix into a single-chain disordered state, and then a mixed solution was incubated at 4° C. for more than 24 hours to fully self-assemble into a well-folded collagen triple helix; and a circular dichroism (CD) experiment was performed by using a Chirascan instrument (Applied Photophysics Ltd, England). Wavelength scanning from 190 nm to 260 nm was performed at 4° C. with an interval of 1 nm per step. A thermal denaturation experiment was performed at 225 nm, where the temperature gradually increased from 4° C. to 80° C. at a gradient heating rate of 1° C./6 min. The Tₘ value is calculated by fitting a first derivative of a thermal denaturation curve.

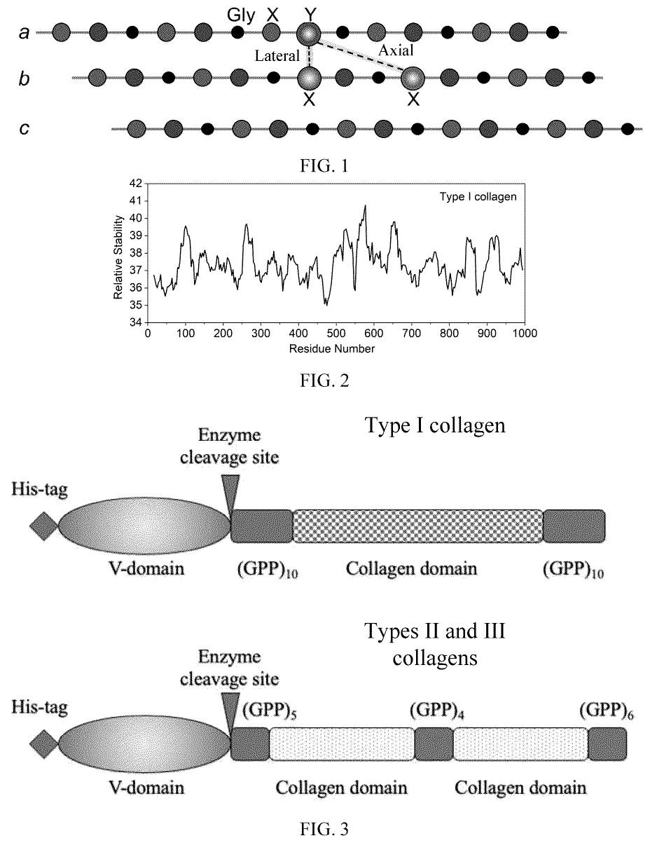

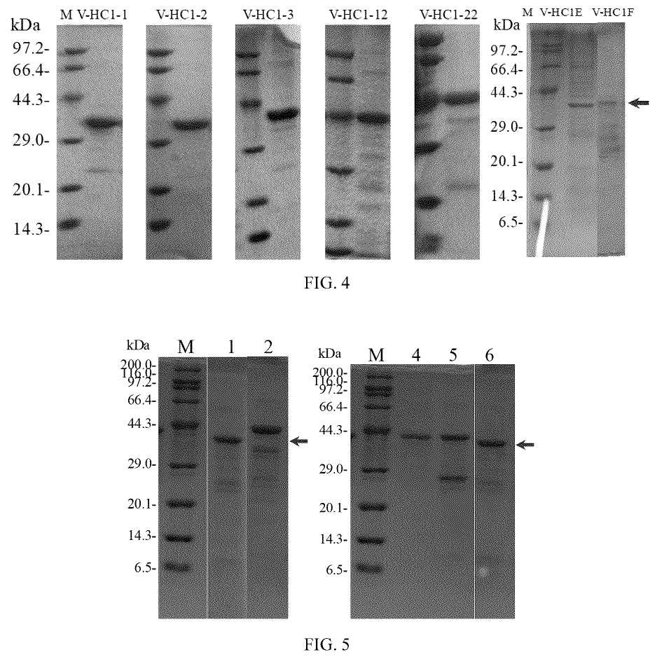

FIG. 2 is a relative stability chart of type I collagen obtained by calculating a thermal stability value of each triplet in a natural human type I collagen sequence and plotting an average relative stability curve according to the above method. As shown in FIG. 2, consecutive triplets with high thermal stability may be truncated to obtain sequence fragments with high thermal stability, or the truncated sequences with high thermal stability are further spliced to obtain a collagen domain sequence.

With reference to the above method, sequence fragments with high thermal stability are truncated from a natural human type I collagen al chain (NCBI accession number NP_000079.2), a human type II collagen al chain (NCBI accession number NP_001835.3), and a human type III collagen al chain (NCBI accession number NP_000081.2), or the truncated sequences with high thermal stability are further spliced to obtain a collagen domain sequence. Collagen domain sequences with predicted Tₘ values of 38-39° C. and high triple helix propensity are selected as target sequences, and sequences with low Tₘ values and low triple helix propensity are selected as control sequences.

Several sequences of different types of collagens with predicted Tₘ values of 38-39° C. are as follows:
(1) Amino acid sequences shown in SEQ ID NO:1-7; (where SEQ ID NO:1-3 are fragments selected from natural human type I collagen or obtained by splicing a plurality of fragments, named HC1-1, HC1-2, and HC1-3 of type I collagen, with predicted Tₘ values of 38.4° C., 38.5° C., and 38.2° C. respectively; SEQ ID NO:4 is a fragment truncated from natural human type II collagen or obtained by splicing a plurality of fragments, named HC2A of type II collagen, with a predicted Tₘ value of 38.3° C.; SEQ ID NO:5-7 are fragments truncated from natural human type III collagen or obtained by splicing a plurality of fragments, named HC3A, HC3B, and HC3C of type III collagen, with predicted Tₘ values of 38.8° C., 38.8° C., and 39.0° C. respectively);
(2) an amino acid sequence obtained by combining any two sequences as shown in SEQ ID NO: 1-3, such as SEQ ID NO:8 (named HC1-12, with a predicted Tₘ value of 38.4° C.) obtained by combining SEQ ID NO:1 and SEQ ID NO:2; and
(3) an amino acid sequence obtained by repeating a sequence as shown in any one of SEQ ID NO: 1-7 for 2-3 times, such as SEQ ID NO:9 (named HC1-22, with a predicted Tₘ value of 38.4° C.) obtained by repeating SEQ ID NO:2 twice.

Several sequences with low predicted Tₘ values (36-37° C.) are as follows: SEQ ID NO: 10-12 (named HC1E, HC1F, and HC2B, with predicted Tₘ values of 37.1° C., 36.3° C., and 36.5° C. respectively).

As shown in Table 1, among SEQ ID NO:1-12, all directly selected sequence fragments are not subjected to sequence modification (100% homology with natural human collagen sequences), and all collagen domain sequences obtained by splicing have more than 57% homology with natural human collagen sequences.

TABLE 1

| Sequence name | Homology with natural sequences (%) |
|---|---|
| HC1-1 | 57.05 |
| HC1-2 | 100 |
| HC1-3 | 62.80 |
| HC2A | 85.59 |
| HC3A | 64.91 |
| HC3B | 65.00 |
| HC3C | 66.95 |
| HC1E | 100 |
| HC1F | 82.35 |
| HC2B | 63.49 |

### Example 2: Collagen Sequence Design

A single protein chain containing the collagen domain of Example 1 is designed. A structure of the single protein chain includes: a folding domain, a repeating sequence module, and a collagen domain.

Introduction of the folding domain assists collagen folding to form a triple helix structure. Optionally, the folding domain is a V-domain or a coiled-coil domain; optionally, an amino acid sequence of the V-domain is as shown in SEQ ID NO:13; and optionally, an amino acid sequence of the coiled-coil domain is as shown in SEQ ID NO: 14.

Introduction of the repeating sequence module assists folding of a collagen triple helix and improves its thermal stability. Optionally, a plurality of repeating sequence modules may be arranged and located at both termini of a collagen domain or both termini of a plurality of collagen domains; and for example, when type II collagen is expressed, a plurality of collagen domains may be arranged, and the plurality of collagen domains are connected through repeating sequence modules. Optionally, sequences of the repeating sequence modules may be identical or different. Optionally, the repeating sequence module employs (GPP)ₙ. Optionally, when a plurality of repeating sequence modules are arranged, values of n in each repeating sequence module (GPP)ₙ may be identical or different; and given that proline at a sequence terminus may be unfavorable for protein expression, an additional glycine is added at a terminus of a collagen amino acid sequence.

As an example, this example designs an amino acid sequence of a single protein chain of collagen with a structure shown in FIG. 10. An amino acid sequence of the folding domain V-domain is as shown in SEQ ID NO:13; and a collagen domain sequence is derived from SEQ ID NO: 1-12 of Example 1.

As an example, as shown in FIG. 3, for sequences derived from type I collagen, short peptides of repeating sequence module (Gly-Pro-Pro)₁₀ (abbreviated as (GPP)₁₀, SEQ ID NO:23) are inserted at both termini of sequences HC1-1, HC1-2, HC1-3, HC1-12, HC1-22, HC1E, and HC1F of Example 1, and after the folding domain V-domain is inserted at the N-terminus, the sequences are named V-HC1-1, V-HC1-2, V-HC1-3, V-HC1-12, V-HC1-22, V-HC1E, and V-HC1F respectively, where the HC1-12 sequence is formed by splicing the HC1-1 sequence and the HC1-2 sequence, and the HC1-22 sequence is formed by splicing two HC1-2 sequences. An amino acid sequence of V-HC1-1 is as shown in SEQ ID NO: 15, and a nucleotide sequence encoding V-HC1-1 is as shown in SEQ ID NO: 16. Amino acid sequences of V-HC1-2, V-HC1-3, V-HC-12, V-HC1-22, V-HC1E, and V-HC1F are obtained by correspondingly replacing the collagen domain sequences in Example 1 on the basis of the amino acid sequence of V-HC1-1.

For sequences derived from types II and III collagens that match natural collagen the morphology, short peptides of (Gly-Pro-Pro)₅, (Gly-Pro-Pro)₄, and (Gly-Pro-Pro)₆ (abbreviated as (GPP)₅ (SEQ ID NO:24), (GPP)₄ (SEQ ID NO:25), and (GPP)₆ (SEQ ID NO:26)) are inserted at the N-terminus, middle, and C-terminus of collagen fragments in sequences HC2A, HC2B, HC3A, HC3B, and HC3C, and the sequences are named V-HC2A, V-HC2B, V-HC3A, V-HC3B, and V-HC3C respectively, with the sequence design shown in FIG. 3. An amino acid sequence of V-HC2A is as shown in SEQ ID NO: 17, and a nucleotide sequence encoding V-HC2A is as shown in SEQ ID NO: 18. An amino acid sequence of V-HC2B is obtained by correspondingly replacing the collagen domain sequence in Example 1 on the basis of the amino acid sequence of V-HC2A. An amino acid sequence of V-HC3A is as shown in SEQ ID NO:19, and a nucleotide sequence encoding V-HC3A is as shown in SEQ ID NO:20. Amino acid sequences of V-HC3B and V-HC3C are obtained by correspondingly replacing the collagen domain sequences in Example 1 on the basis of the amino acid sequence of V-HC3A.

### Example 3: Construction of Recombinant Plasmids and Recombinant Strain

When a nucleotide sequence of a single protein chain (such as the single protein chain of Example 2) was synthesized, a GC base was introduced at a 5′ flanking end, and Nco I and Bam HI enzyme cleavage sites were introduced at 5′ and 3′ ends respectively. Subsequently, the above synthesized genes were inserted between Nco I and Bam HI of a pColdIII-M plasmid to obtain corresponding recombinant collagen plasmids, where the pColdIII-M plasmid was obtained by mutating a Nde I enzyme cleavage site on the pColdIII plasmid to an Nco I enzyme cleavage site. Correctly sequenced recombinant plasmids were transformed into E. coli BL21 (DE3) competent cells respectively, spread on LB plates containing ampicillin for culture and screening, and preserved in glycerol tubes to obtain recombinant strain containing recombinant collagen.

### Example 4: Expression, Purification and Enzyme Cleavage Optimization of Collagen Sequences

Fermentation culture of the recombinant strain obtained in Example 3 was performed in a shake flask, after bacterial cells were collected, broken and centrifuged, a supernatant was collected with 5 mL of His Trap™ HP for affinity purification, and samples were collected at imidazole concentrations of 175 mmol/L and 400 mmol/L, where SDS-PAGE identification results of the samples are shown in FIGS. 4 and 5. Theoretical molecular weights of V-HC1-1, V-HC1-2, V-HC1-3, V-HC1-12, V-HC1-22, V-HC1E, and V-HC1F are 25.13 kDa, 24.81 kDa, 28.15 kDa, 34.38 kDa, 34.33 kDa, 25.15 kDa, and 26.09 kDa respectively, and their apparent molecular weights on SDS-PAGE are about 36 kDa, 35 kDa, 40 kDa, 48 kDa, 48 kDa, 37 kDa, and 38 kDa respectively (as shown in FIG. 4); and theoretical molecular weights of V-HC2A, V-HC2B, V-HC3A, V-HC3B, and V-HC3C are 34.32 kDa, 37.15 kDa, 34.70 kDa, 34.28 kDa, and 32.61 kDa respectively, and their apparent molecular weights on SDS-PAGE are about 37 kDa, 44 kDa, 43 kDa, 43 kDa, and 38 kDa respectively (as shown in FIG. 5), which are about 1.4 times the theoretical molecular weights, maybe because high content of proline in a collagen sequence results in a slower migration rate of target protein on SDS-PAGE than proteins of the same molecular weight, which is consistent with literature reports.

Removal of a folding domain is a prerequisite for self-assembly of collagen molecules through transverse and head-to-tail staggered arrangement, and which ultimately promotes the formation of striated fibrils. Therefore, during sequence design, an LVPRGS sequence of trypsin enzyme cleavage site is introduced between a collagen structural domain and a folding domain, so the folding domain may be removed by adding an appropriate amount of trypsin for digestion to obtain a pure collagen domain structure. Under the action of trypsin, the V-domain is digested into a plurality of short peptides containing 2-20 amino acid residues, and when the collagen structural domain is correctly folded to form a rigid triple helix structure under the action of the V-domain, the V-domain is not digested by trypsin in a short time.

V-HC1-2 is selected as a pattern protein to optimize trypsin digestion conditions. The results show that when a molar ratio is 20:1 and digestion is performed for 3 h, the V-domain and a miscellaneous protein are basically completely digested, and only one band has a molecular weight of about 25 kDa that corresponds to 1.4 times a molecular weight of an HC1-2 collagen domain; after 12 h, the band becomes lighter, maybe because too long digestion in a high-concentration trypsin solution results in the cleavage of a small portion of the triple helix; when the molar ratio is 200:1, some bands are still undigested at 3 h but disappear after 6 h, indicating that the V-domain is basically removed at this time, and the bands are not significantly lightened within 12 h; when the molar ratio is 2000:1, the V-domain is not completely cleaved after 9 h of enzyme cleavage; and the band gradually disappears before enzyme cleavage after about 12 h. According to enzyme cleavage results, a molar ratio of 200:1 is selected for enzyme cleavage, and an enzyme cleavage duration is controlled to be 6-12 h.

### Example 5: SDS-PAGE Identification and Analysis of Collagen after Enzyme Cleavage

Under optimal enzyme cleavage optimization conditions of Example 4, enzyme cleavage was performed for five types of collagens. The results show that after trypsin digestion, collagens V-HC1-1, V-HC1-2, V-HC1-3, and V-HC1-22 all form a single band with a purity reaching electrophoretic purity, and an apparent molecular weight corresponding to 1.4 times a theoretical molecular weight after enzyme cleavage.

### Example 6: Formation of Collagen with a Triple Helix Structure, and Circular Dichroism Characterization of Sequence

To confirm a secondary structure of a collagen domain, a freeze-dried collagen sample obtained after enzyme cleavage and desalting in Example 5 was dissolved in a 10 mmol/L sodium phosphate buffer to form a solution with a concentration of 1 mg/mL, and the solution was equilibrated at 4° C. for 48 h. After equilibration, full-wavelength scanning was performed based on circular dichroism.

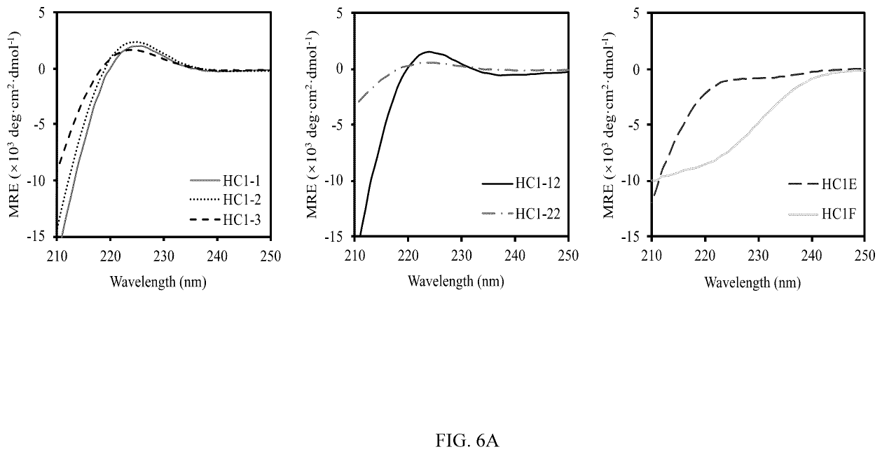

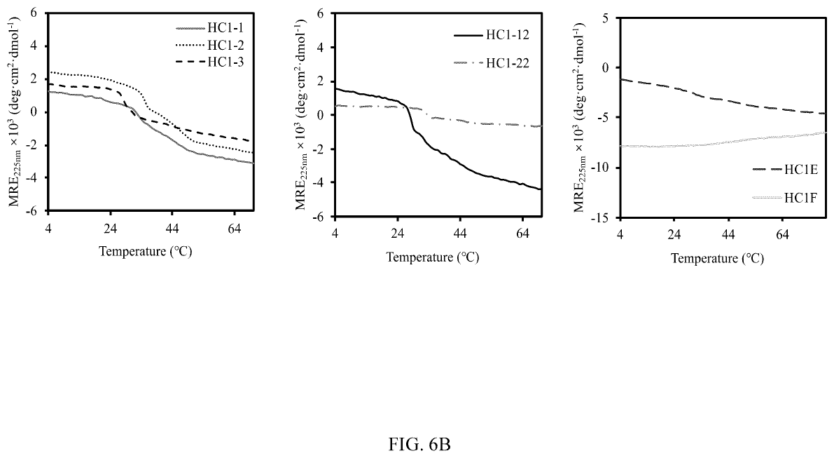

For the design of human type I collagen, as shown in FIG. 6A, HC1-1, HC1-2, and HC1-3 all exhibit characteristic positive absorption peaks at 225 nm, indicating that all three types of collagens are properly folded to form a triple helix structure with the assistance of a V-domain. In contrast, control fragments HC1E and HC1F with low predicted Tₘ values exhibit no characteristic positive absorption peaks at 225 nm, indicating that they cannot be properly folded to form a triple helix structure. A thermal denaturation temperature of the collagen domain was further measured by circular dichroism, predicted Tₘ values of HC1-1, HC1-2, and HC1-3 were 38.4° C., 38.5° C., and 38.2° C. respectively, and as shown in FIG. 6B, thermal denaturation curves at 225 nm from 4° C. to 70° C. were detected by circular dichroism, and the thermal denaturation curves were fitted (see Table 1); and results show that Tₘ values of HC1-1, HC1-2, and HC1-3 are 37.2° C., 38.7° C., and 32.4° C. respectively, while predicted Tₘ values of control fragments HC1E and HC1F with low predicted Tₘ are 37.1° C. and 36.3° C. respectively, and thermal transition of HC1E and HC1F cannot be measured according to thermal denaturation curves detected by circular dichroism at 225 nm from 4° C. to 70° C., indicating that HC1E and HC1F are not properly folded to form a triple helix structure.

Additionally, collagens HC1-12 and HC1-22 obtained by combining fragments 1 and 2 with high thermal stability may further be properly folded to form a triple helix structure; and predicted Tₘ values of HC1-12 and HC1-22 are 38.4° C. and 38.4° C. respectively, and Tₘ values of HC1-12 and HC1-22 detected by circular dichroism are 33.0° C. and 33.6° C. respectively, indicating that elongation of the collagen domain leads to a certain decline in thermal stability. Analysis suggests that collagen sequence lengthening and splicing of two sequence segments result in that an assisting force of the V-domain for triple helix folding is insufficient to transmit from the N-terminus to the farther C-terminus, which leads to insufficient rigidity and looseness of a triple helix formed in some regions, and fast unfolding, thereby reducing the thermal stability.

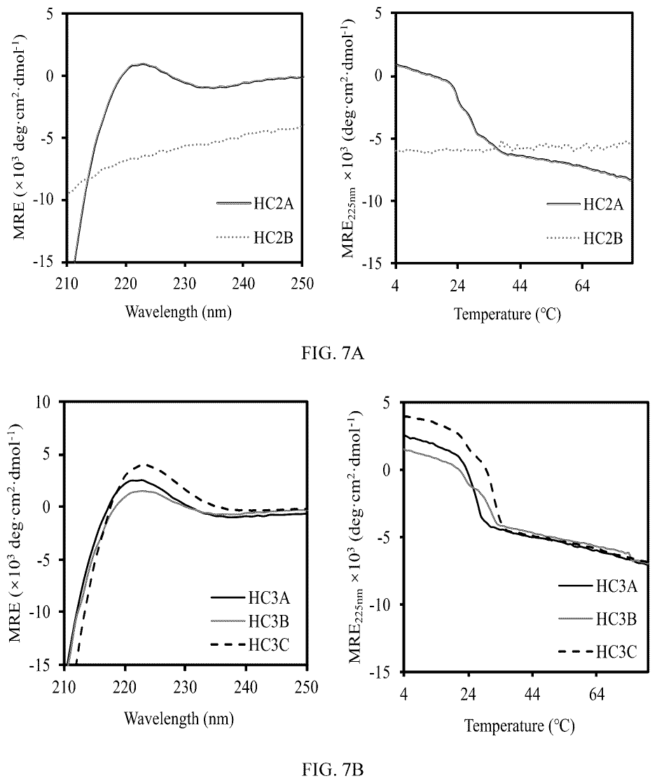

For the design of human types II and III collagens, to confirm a secondary structure of a collagen domain, full-wavelength scanning was performed based on circular dichroism, and as shown in FIGS. 7A and 7B, HC2A, HC3A, HC3B, and HC3C all exhibit characteristic positive absorption peaks at 225 nm, indicating that all four types of collagens are properly folded to form a triple helix structure with the assistance of the V-domain. In contrast, a control fragment HC2B with a low predicted Tₘ value exhibits no characteristic positive absorption peak at 225 nm, indicating that the fragment cannot be properly folded to form a triple helix structure. A thermal denaturation temperature of the collagen domain was further measured by circular dichroism, predicted Tₘ values of HC2A, HC3A, HC3B, and HC3C were 38.3° C., 38.8° C., 38.8° C., and 39.0° C. respectively, and a predicted Tₘ value of HC2B with low predicted stability was 36.5° C. As shown in FIGS. 7A and 7B, thermal denaturation curves at 225 nm from 4° C. to 70° C. were detected by circular dichroism, and the thermal denaturation curves were fitted (see Table 2); and results show that Tₘ values of HC2A, HC3A, HC3B, and HC3C are 28.2° C., 25.1° C., 28.2° C., and 30.3° C. respectively, and the thermal transition of the fragment HC2B with a low predicted Tₘ value cannot be measured, indicating that HC2B is not properly folded to form a triple helix structure.

The above results indicate that all the designed collagen fragments with high thermal stability and predicted Tₘ of 38-39° C. may be properly folded to form a triple helix structure, while the fragments with low thermal stability and predicted Tₘ of less than 38° C. cannot be properly folded, indicating that collagen fragments with different thermal stabilities may be effectively designed by calculating and predicting the thermal stability of human collagen, while achieving heterologous expression thereof in Escherichia coli.

TABLE 2
Predicted and fitted Tₘ of human collagen

| | Sample | Predicted Tₘ (° C.) | Fitted Tₘ (° C.) |
|---|---|---|---|
| Type I | HC1-1 | 38.4 | 37.2 |
| collagen | HC1-2 | 38.5 | 38.7 |
| | HC1-3 | 38.2 | 32.4 |
| | HC1E | 37.1 | / |
| | HC1F | 36.3 | / |
| | HC1-12 | 38.4 | 33.0 |
| | HC1-22 | 38.4 | 33.6 |
| Type II | HC2A | 38.3 | 28.2 |
| collagen | HC2B | 36.5 | / |
| Type III | HC3A | 38.8 | 25.1 |
| collagen | HC3B | 38.8 | 28.2 |
| | HC3C | 39.0 | 30.3 |

### Example 7: Collagen Fibers Formed by High-Polymerization-Induced Self-Assembly of Collagen (Characterization of Collagen Sequence Self-Assembly Morphology)

To observe whether a collagen domain self-assembles into a higher-order structure in a high-concentration solution, freeze-dried type I collagens HC1-1, HC1-2, HC1-3, HC1-12, and HC1-22 of sequences in Example 1 were dissolved in a 10 mmol/L sodium phosphate buffer to prepare a solution with a concentration of 0.5 mmol/L, and then negative staining was performed after assembly at 4° C. for 4 days, followed by TEM characterization of morphology.

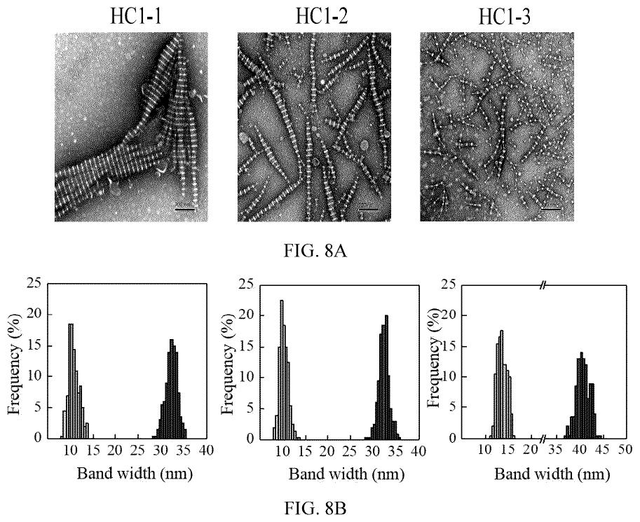

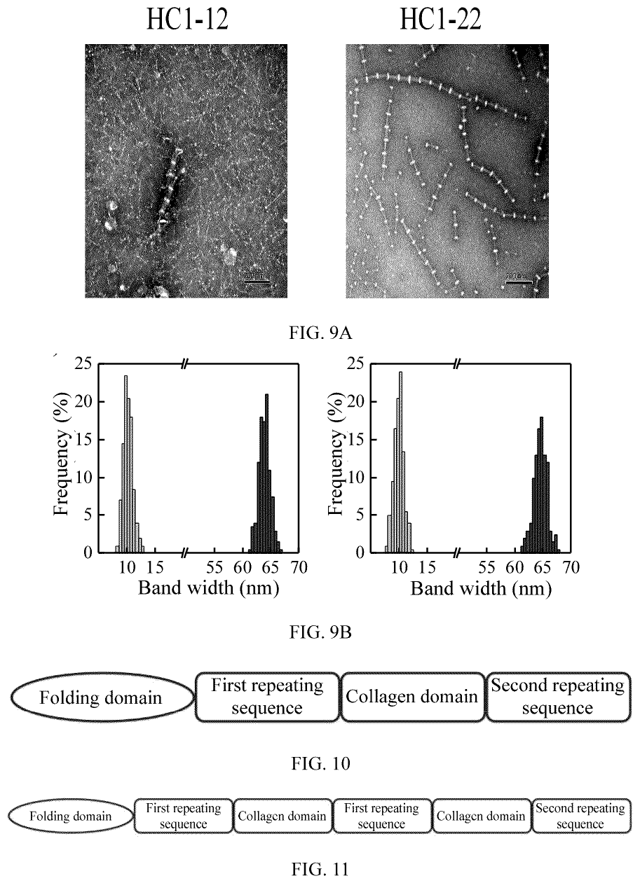

As shown in FIGS. 8A, 8B, 9A, and 9B, banded fibers with periodic alternating light and dark stripes may be observed in the field of view, which are similar to fibers of natural type I collagen in morphology, indicating that collagen domains of designed collagen all self-assemble to form biomimetic microfiber structures. Literature reports indicate that a length of each Gly-Pro-Pro triplet is 1.0 nm, and a length of each XYGs triplet is 0.9 nm. Results of measuring lengths of light and dark stripes by using ImageJ (see Table 3) show that lengths of light stripes of HC1-1, HC1-2, HC1-3, HC1-12, and HC1-22 are about 10.6 nm, 10.3 nm, 11.7 nm, 10.2 nm, and 9.9 nm respectively, which correspond to a theoretical length of 10 nm of a (GPP)₁₀ repeating sequence module; and lengths of dark stripes are about 32.2 nm, 32.3 nm, 42.8 nm, 63.8 nm, and 64.5 nm respectively, which are consistent with theoretical values.

Additionally, it can be observed from FIGS. 8A, 8B, 9A, and 9B, the number of banded fibers assembled by HC1-22 in the field of view is greater than that of HC1-12, indicating that a self-assembly effect of HC1-22 is superior to that of HC1-12, maybe due to higher thermal stability of HC1-2 than HC1-1, which compromises the assembly effect. Additionally, results show that fewer banded fibers may be observed in HC1-12 and HC1-22 whose self-assembly effects are not as good as those of shorter HC1-1 and HC1-2 in terms of fiber length or fiber aggregation morphology.

TABLE 3
Statistics of band widths of collagen fibers

| Sequence | Measured band width (nm) | | Theoretical band width (nm) | |
|---|---|---|---|---|
| name | Light stripe | Dark stripe | Light stripe | Dark stripe |
| HC1-1 | 10.6 ± 1.2 | 32.2 ± 1.3 | 10 | 32.4 |
| HC1-2 | 10.3 ± 0.9 | 32.3 ± 1.2 | 10 | 32.4 |
| HC1-3 | 11.7 ± 1.1 | 42.8 ± 1.6 | 10 | 43.2 |
| HC1-12 | 10.2 ± 0.8 | 63.8 ± 1.0 | 10 | 63.9 |
| HC1-22 | 9.9 ± 0.7 | 64.5 ± 1.2 | 10 | 64.8 |

### Example 8: Construction of Collagens with Different Repeating Sequences, and Performance Detection

1. Construction of Single Collagen Chains with Different Repeating Sequences

On the basis of Example 2, amino acid sequences of a first repeating sequence module and a second repeating sequence module in a single collagen chain with a structure shown in FIG. 10 were modified, and taking a collagen domain HC1-2 (an amino acid sequence as shown in SEQ ID NO: 2), a folding domain V-domain (an amino acid sequence as shown in SEQ ID NO:13), and an enzyme cleavage site LVPRGSP (an amino acid sequence as shown in SEQ ID NO:21) as an example, collagens with different repeating sequences were constructed.

Specific structures are as follows (m=1):
(1) The first repeating sequence module was modified to KD3 (an amino acid sequence as shown in SEQ ID NO:28), and an amino acid sequence of the second repeating sequence module was modified to P10 (an amino acid sequence as shown in SEQ ID NO:23), with a collagen structure as follows: a folding domain, {KD3, HC1-2}m, and P10, where a complete amino acid sequence is shown in SEQ ID NO:29;
(2) the first repeating sequence module was modified to P10, and an amino acid sequence of the second repeating sequence module was modified to KD3, with a collagen structure as follows: a folding domain, {P10, HC1-2}m, and KD3, where a complete amino acid sequence is shown in SEQ ID NO:30;
(3) the first repeating sequence module was modified to P10, and an amino acid sequence of the second repeating sequence module was modified to KD2 (an amino acid sequence as shown in SEQ ID NO:27), with a collagen structure as follows: a folding domain, {P10, HC1-2}m, and KD2, where a complete amino acid sequence is shown in SEQ ID NO:31;
(4) the first repeating sequence module was modified to KD2, and an amino acid sequence of the second repeating sequence module was modified to KD2, with a collagen structure as follows: a folding domain, {KD2, HC1-2}m, and KD2, where a complete amino acid sequence is shown in SEQ ID NO:33; and
(5) the first repeating sequence module was modified to P10, and an amino acid sequence of the second repeating sequence module was modified to P10, with a collagen structure as follows: a folding domain, {P10, HC1-2}m, and P10, where a complete amino acid sequence is shown in SEQ ID NO:32.

Collagens prepared respectively according to the method of Example 3 are named V-KD3BP10, V-P10BKD3, V-P10BKD2, V-KD2BKD2, and V-P10BP10 according to amino acid sequences of the first repeating sequence module and the second repeating sequence module, where “B” represents a collagen domain (collagen domain HC1-2 in this example), “V” represents a folding domain V-domain, “P10” represents (GPP)₁₀ with an amino acid sequence as shown in SEQ ID NO:23, “KD2” has an amino acid sequence as shown in SEQ ID NO:27, “KD3” has an amino acid sequence as shown in SEQ ID NO:28, and amino acid sequences of the prepared collagens are shown in SEQ ID NO:29-SEQ ID NO:33.

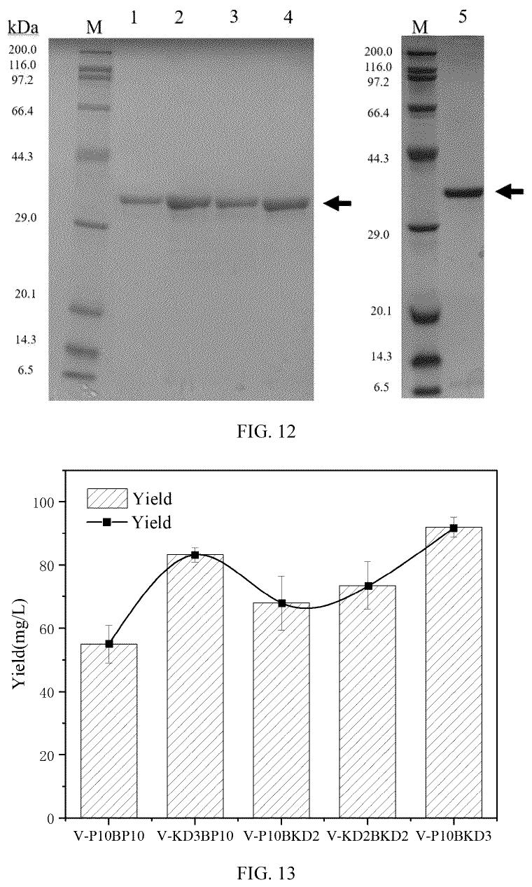

Enzymatically cleaved collagens KD3BP10, P10BKD3, P10BKD2, KD2BKD2, and P10BP10 were prepared through expression, purification and enzyme cleavage optimization of collagens according to the method described in Example 4. Results of SDS-PAGE verification according to the method of Example 5 are shown in FIG. 12.

Results of yields obtained after desalting, freeze-drying, and weighing verified proteins are shown in FIG. 13. Yields of recombinant collagens V-P10BP10, V-KD3BP10, V-P10BKD2, V-KD2BKD2, and V-P10BKD3 are 55.02 mg/L, 83.25 mg/L, 68.02 mg/L, 73.51 mg/L, and 91.98 mg/L, respectively, and the yields of collagens modified in repeating sequences (V-KD3BP10, V-P10BKD2, V-KD2BKD2, and V-P10BKD3) increase by 23.6%-67.18% compared with that of V-P10BP10, with V-P10BKD3 having a highest yield.

### 2. Circular Dichroism Characterization of Collagens and Sequence Structures

Secondary structures of collagen domains of collagens KD3BP10, P10BKD3, P10BKD2, KD2BKD2, and P10BP10 prepared above were detected and confirmed. The collagens were freeze-dried and dissolved in a 10 mmol/L sodium phosphate buffer to form a solution with a concentration of 1 mg/mL, and the solution was equilibrated at 4° C. for 24 h. After equilibration, full-wavelength scanning was performed based on circular dichroism.

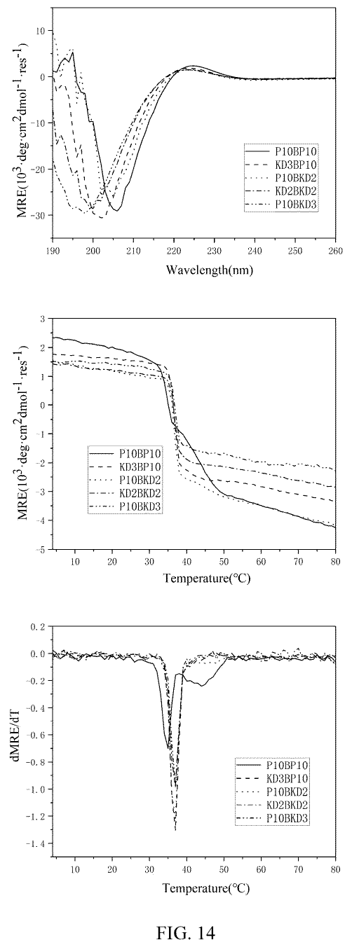

As shown in FIG. 14, P10BP10, KD3BP10, P10BKD2, KD2BKD2, and P10BKD3 all exhibit characteristic positive absorption peaks at 225 nm, indicating that all six types of collagens are properly folded to form a triple helix structure with the assistance of a V-domain. Thermal denaturation curves at 225 nm from 4° C. to 80° C. were detected by circular dichroism, and Tₘ values of collagens were obtained by first-order derivation of the thermal denaturation curves, where a Tₘ value of P10BP10 was 35° C., and Tₘ values of other sequences were all 37° C.

The above results indicate that the collagen sequences designed in the present disclosure all are properly folded to form a triple helix structure, and the Tₘ value increases by 2° C. compared with that of a P10BP10 collagen sequence, indicating that all the designed sequences enhance the thermal stability of proteins.

3. Salt Tolerance Detection of Collagens with Different Repeating Sequences

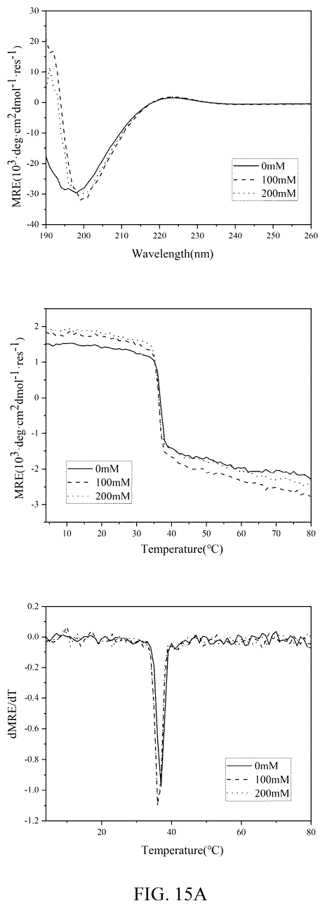

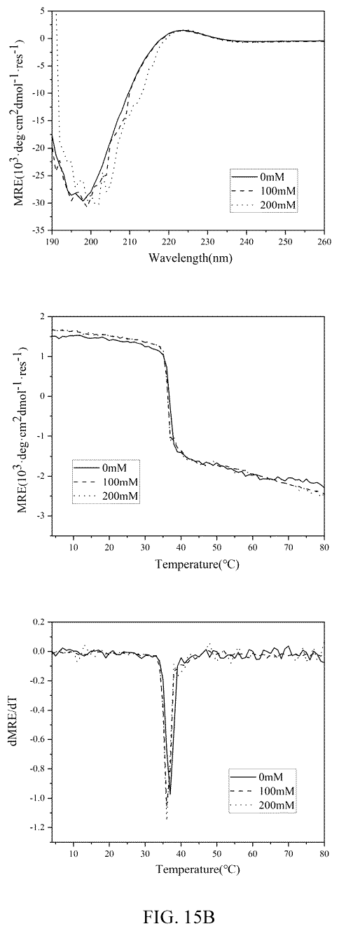

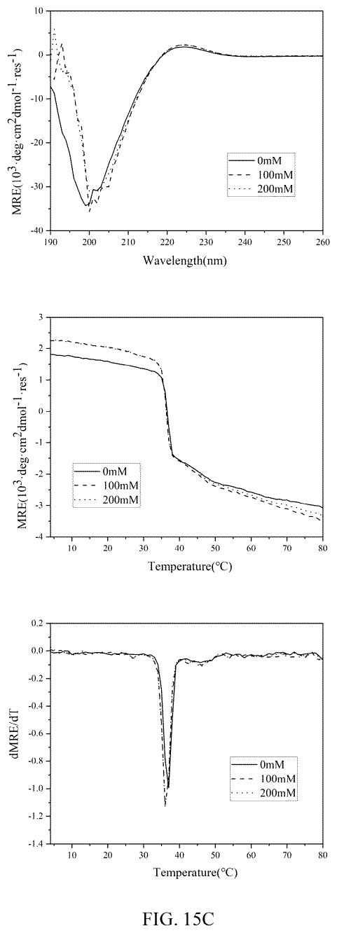

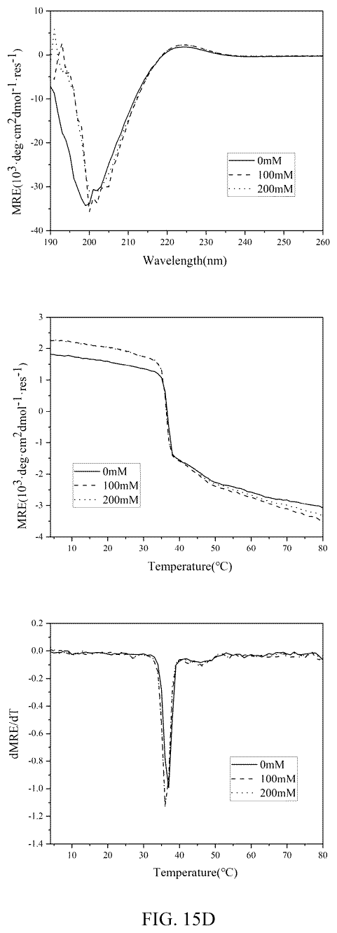

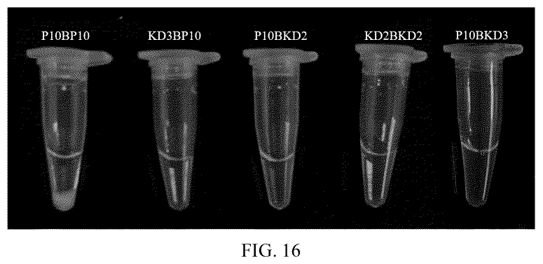

Stabilities of the collagens KD3BP10, P10BKD3, P10BKD2, KD2BKD2, and P10BP10 prepared above in high-concentration salt solutions was detected. The collagens were freeze-dried and dissolved in a 10 mmol/L sodium phosphate buffer to form a solution with a concentration of 1 mg/mL. On this basis, 100 mmol and 200 mmol of sodium chloride were added to the solution, with results shown in FIGS. 15A, 15B, 15C, and 15D. Thermal denaturation curves at 225 nm from 4° C. to 80° C. were detected by circular dichroism, and Tₘ values of collagens were obtained by first-order derivation of the thermal denaturation curves. The results show that the Tₘ value of each sequence is still 37° C. in a high-salt environment, indicating that the modified collagens still have good salt tolerance.

4. Solubility Detection of Collagens with Different Repeating Sequences

Solubilities of the collagens KD3BP10, P10BKD3, P10BKD2, KD2BKD2, and P10BP10 prepared above was detected. The collagens were freeze-dried and dissolved in a 10 mmol/L sodium phosphate buffer to prepare a solution with a sample concentration of 2 mmol/L, and the solution was allowed to stand undisturbed at 4° C. for 1 day, with results shown in FIG. 16, where P10BP10 precipitated, while other proteins had no precipitates. This indicates that the modified collagens have lower turbidity and higher molecular solubility than the P10BP10 sequence at a same concentration.

5. Performance Detection of Single Collagen Chains Prepared from Collagen Domains with Different Amino Acid Sequences

On the basis of “1. Construction of single collagen chains with different repeating sequences”, the collagen domain HC1-2 (an amino acid sequence as shown in SEQ ID NO:2) was further modified to HC1-1 (an amino acid sequence as shown in SEQ ID NO:1), HC1-3 (an amino acid sequence as shown in SEQ ID NO:3), HC2A (an amino acid sequence as shown in SEQ ID NO: 4), HC3A (an amino acid sequence as shown in SEQ ID NO:5), HC3B (an amino acid sequence as shown in SEQ ID NO:6), HC3C (an amino acid sequence as shown in SEQ ID NO:7), HC1-12 (an amino acid sequence as shown in SEQ ID NO:8), and HC1-22 (an amino acid sequence as shown in SEQ ID NO:9) to obtain a plurality of single collagen chains, and the single collagen chains were expressed and purified to analyze the versatility of the repeating sequence modification method of the present disclosure in improving the collagen yield, heat resistance, and salt tolerance.

The results show that the yields of the above single collagen chains increase by more than 15% than (GPP)₁₀; and purified collagens have a heat-resistant temperature Tₘ increased by about 2° C. or maintained at about 37-38° C., and have good salt tolerance.

### Example 9: Collagen-Containing Products

A collagen-containing product may be a product in the fields of beauty, chemical engineering, food health, medical treatment/biomedicine, cosmetics, and feed, such as beauty cosmetics (a facial mask, an essence, a cream, or the like), an artificial collagen casing, a nutritional health product (a collagen powder, an oral liquid), a medical dressing, a hemostatic material, an artificial bone scaffold, an injectable filler, an artificial blood vessel, an eye drop, a sustained-release drug carrier, or the like.

In the collagen-containing product, the collagen has a collagen domain sequence of Example 1 of the present disclosure, or a collagen sequence of Example 2, or an enzymatically cleaved collagen sequence obtained in Example 4, or an enzymatically cleaved collagen sequence obtained in Example 8.

Further, the collagen is a collagen expressing a triple helix structure.

Further, the collagen is a type I, type II or type III collagen.

Further, other components, formulations, and preparation processes of the above collagen-containing product may be achieved by those skilled in the art through any method in the prior art.

Optionally, the product is a dressing, including a collagen membrane layer containing the triple helix structure of the present disclosure; optionally, a drug-loaded cellulose membrane layer is further included; and optionally, the number of the collagen membrane layers is 2-4, the number of the drug-loaded cellulose membrane layers is 1-3, and one drug-loaded cellulose membrane layer is correspondingly disposed between two collagen membrane layers.

Optionally, the product is an injectable filler, and is prepared by the following steps: the collagen of the present disclosure was dissolved in water for injection to form a collagen solution with a mass fraction of 1.0%-5.0%, medical-grade sodium hyaluronate was dissolved in water for injection to form a medical-grade sodium hyaluronate solution with a mass fraction of 0.5%-2.0%, the two solutions were mixed in a certain ratio, then a certain amount of medical-grade hydroxyapatite was added before mixing and stirring, an appropriate amount of medical-grade glycerol was added during stirring, and vacuum degassing was performed after mixing uniformly, to obtain a finished product.

Optionally, the product is an artificial bone scaffold material, which is prepared by the following steps: the collagen of the present disclosure was configured into a collagen solution with a certain concentration, then a pH value to 6.5-7.5 was adjusted, transglutaminase and nano-hydroxyapatite were added, a resulting mixture was transferred to a mold for reaction for a period of time, and cooling, enzyme inactivating, and freeze-drying were performed to obtain the artificial bone scaffold material.

Sequences involved in the present disclosure
SEQ ID NO: 1: an amino acid sequence of HC1-1
GARGLPGTAGLPGMKGHRGFPGERGLDGAKGDAGPAGPKGEPGSPGENGAPG
QMGPRGPQGPPGPPGPKGNSGEPGAPGSKGDTGAKGEPGPVGVQGPPGPAGEEGKR
SEQ ID NO: 2: an amino acid sequence of HC1-2
GFPGERGVQGPPGPAGPRGANGAPGNDGAKGDAGAPGAPGSQGAPGLQGMPG
ERGAAGLPGPKGDRGDAGPKGADGSPGKDGVRGLTGPIGPPGPAGAPGDKGESGPS
SEQ ID NO: 3: an amino acid sequence of HC1-3
GPAGFAGPPGADGQPGAKGEPGDAGAKGDAGPPGPAGPAGPPGPIGESGREGAP
GAEGSPGRDGSPGAKGDRGETGPAGPPGFPGERGAPGPAGPAGPVGPVGARGPAGPQGP
RGDKGETGEQGDRGIKGHRGFSGLQ
SEQ ID NO: 4: an amino acid sequence of HC2A
GLTGPAGEPGREGSPGADGPPGRDGAAGVKGDRGETGAVGAPGAPGPPGDRGE
AGAQGPMGPSGPAGARGIQGPQGPRGDKGEAGEPGERGLKGHRGFTGLQGLPGPPGPS
SEQ ID NO: 5: an amino acid sequence of HC3A
GFPGMKGHRGFDGRNGEKGETGAPGLKGENGLPGENGAPGPMGPRGAPGERG
SPGPKGDKGEPGPPGADGVPGKDGPRGPTGPIGPPGPAGQPGDKGEP
SEQ ID NO: 6: an amino acid sequence of HC3B
GFPGMKGHRGFDGRNGEKGETGAPGLKGENGLPGENGAPGPMGPRGAPGERG
AKGEPGPRGERGEAGIPGVPGAKGEDGKPGEPGPKGDAGAPGAPGPKGDAGAPGER
SEQ ID NO: 7: an amino acid sequence of HC3C
GFPGMKGHRGFDGRNGEKGETGAPGLKGENGLPGENGAPGPMGPRGAPGERG
AKGEPGPRGERGEAGIPGVPGAKGEDGRDGNPGSDGLPGRDGSPGPKGDRGENGSP
SEQ ID NO: 8: an amino acid sequence of HC1-12
GARGLPGTAGLPGMKGHRGFPGERGLDGAKGDAGPAGPKGEPGSPGENGAPG
QMGPRGPQGPPGPPGPKGNSGEPGAPGSKGDTGAKGEPGPVGVQGPPGPAGEEGKRGF
PGERGVQGPPGPAGPRGANGAPGNDGAKGDAGAPGAPGSQGAPGLQGMPGERGAAGL
PGPKGDRGDAGPKGADGSPGKDGVRGLTGPIGPPGPAGAPGDKGESGPS
SEQ ID NO: 9: an amino acid sequence of HC1-22
GFPGERGVQGPPGPAGPRGANGAPGNDGAKGDAGAPGAPGSQGAPGLQGMPG
ERGAAGLPGPKGDRGDAGPKGADGSPGKDGVRGLTGPIGPPGPAGAPGDKGESGPSGFP
GERGVQGPPGPAGPRGANGAPGNDGAKGDAGAPGAPGSQGAPGLQGMPGERGAAGLP
GPKGDRGDAGPKGADGSPGKDGVRGLTGPIGPPGPAGAPGDKGESGPS
SEQ ID NO: 10: an amino acid sequence of HC1E
GPMGPSGPRGLPGPPGAPGPQGFQGPPGEPGEPGASGPMGPRGPPGPPGKNGDD
GEAGKPGRPGERGPPGPQGARGLPGTAGLPGMKGHRGFSGLDGAKGDAGPAGPK
SEQ ID NO: 11: an amino acid sequence of HC1F
GPRGLPGPPGAPGPQGFQGPPGEPGEPGASGPMGPRGPPGPPGKNGDDGEAGKP
GRPGERGPPGPQGARGLPGTAGLPGMKGPAGSPGFQGLPGPAGPPGEAGKPGEQGVPGD
LGAPGPS
SEQ ID NO: 12: an amino acid sequence of HC2B
GANGDPGRPGEPGLPGARGLTGRPGDAGPQGKVGPSGAPGEDGRPGPPGPQGA
RGQPGVMGFPGPKGANGEPGKAGEKGLPGAPGLRGLPGKDGETGAAGERGSPGAQGL
QGPRGLPGTPGTDGPK
SEQ ID NO: 13: an amino acid sequence of V-domain
ADEQEEKAKVRTELIQELAQGLGGIEKKNFPTLGDEDLDHTYMTKLLTYLQERE
QAENSWRKRLLKGIQDHALD
SEQ ID NO: 14: an amino acid sequence of coiled-coil domain
GEIAAIKQEIAAIKKEIAAIKWEIAAIKQGYG
SEQ ID NO: 15: an amino acid sequence of V-HC1-1
HHHHHHADEQEEKAKVRTELIQELAQGLGGIEKKNFPTLGDEDLDHTYMTKLL
TYLQEREQAENSWRKRLLKGIQDHALDLVPRGSPGPPGPPGPPGPPGPPGPPGPPGPPGPP
GPPGARGLPGTAGLPGMKGHRGFPGERGLDGAKGDAGPAGPKGEPGSPGENGAPGQM
GPRGPQGPPGPPGPKGNSGEPGAPGSKGDTGAKGEPGPVGVQGPPGPAGEEGKRGPPGP
PGPPGPPGPPGPPGPPGPPGPPGPPG
SEQ ID NO: 16: a nucleotide sequence of V-HC1-1
CACCATCACCATCACCACGCCGACGAGCAAGAAGAAAAGGCCAAAGTTCGC
ACCGAGCTGATTCAAGAACTGGCGCAAGGTCTGGGCGGCATCGAAAAGAAAAACTT
CCCGACGCTGGGCGATGAAGATCTGGACCACACCTACATGACGAAGCTGCTGACCTA
TCTGCAAGAACGTGAACAAGCCGAGAATAGCTGGCGCAAACGTCTGCTGAAAGGCA
TCCAAGATCATGCGCTGGATCTGGTGCCACGTGGCAGCCCGGGCCCGCCGGGCCCGC
CGGGCCCACCGGGTCCACCGGGCCCGCCGGGCCCACCGGGTCCGCCGGGTCCGCCG
GGTCCGCCGGGCCCACCGGGCGCCCGTGGTCTGCCGGGCACCGCCGGTCTGCCGGG
CATGAAAGGCCATCGCGGTTTCCCGGGTGAACGTGGTCTGGATGGCGCCAAAGGTGA
TGCGGGTCCAGCCGGTCCGAAAGGCGAACCGGGCAGCCCGGGCGAAAATGGTGCGC
CGGGCCAGATGGGTCCGCGTGGTCCACAAGGCCCGCCGGGCCCACCGGGCCCGAAA
GGCAATAGCGGTGAACCGGGCGCCCCGGGCAGTAAAGGCGATACCGGTGCGAAAGG
TGAACCGGGCCCGGTTGGTGTTCAAGGCCCACCGGGCCCAGCGGGTGAAGAAGGTA
AACGTGGTCCGCCGGGTCCACCGGGTCCACCGGGTCCACCGGGCCCACCGGGCCCG
CCGGGCCCACCGGGTCCGCCGGGCCCGCCGGGCCCACCGGGCTAA
SEQ ID NO: 17: an amino acid sequence of V-HC2A
HHHHHHADEQEEKAKVRTELIQELAQGLGGIEKKNFPTLGDEDLDHTYMTKLL
TYLQEREQAENSWRKRLLKGIQDHALDLVPRGSPGPPGPPGPPGPPGPPGLTGPAGEPGR
EGSPGADGPPGRDGAAGVKGDRGETGAVGAPGAPGPPGDRGEAGAQGPMGPSGPAGA
RGIQGPQGPRGDKGEAGEPGERGLKGHRGFTGLQGLPGPPGPSGPPGPPGPPGPPGLTGP
AGEPGREGSPGADGPPGRDGAAGVKGDRGETGAVGAPGAPGPPGDRGEAGAQGPMGP
SGPAGARGIQGPQGPRGDKGEAGEPGERGLKGHRGFTGLQGLPGPPGPSGPPGPPGPPGP
PGPPGPPG
[SEQ ID NO: 18: a nucleotide sequence of V-HC2A
CATCACCATCACCATCATGCGGATGAACAAGAAGAAAAAGCGAAAGTGCGC
ACCGAACTGATTCAAGAACTGGCGCAAGGCCTGGGCGGCATTGAAAAAAAAAACTT
TCCGACCCTGGGCGATGAAGATCTGGATCATACCTATATGACCAAACTGCTGACCTAT
CTGCAAGAACGCGAACAAGCGGAAAACAGCTGGCGCAAACGCCTGCTGAAAGGCAT
TCAAGATCACGCCCTGGACTTAGTGCCGCGCGGTAGCCCGGGTCCGCCGGGTCCGCC
GGGCCCGCCGGGTCCGCCGGGTCCGCCGGGCTTAACCGGCCCGGCCGGCGAACCGG
GCCGTGAGGGCAGCCCGGGCGCCGATGGCCCGCCGGGCCGCGACGGCGCGGCCGGC
GTGAAGGGCGATCGTGGCGAAACGGGCGCGGTGGGTGCGCCGGGTGCGCCGGGCCC
GCCGGGCGATCGTGGTGAAGCGGGCGCCCAAGGCCCAATGGGCCCAAGTGGTCCGG
CGGGTGCGCGCGGCATCCAAGGCCCGCAAGGCCCGCGCGGTGACAAAGGCGAAGCG
GGCGAACCGGGCGAACGTGGCTTAAAAGGCCACCGCGGCTTTACGGGTCTGCAAGG
TTTACCGGGTCCGCCGGGTCCAAGTGGTCCACCGGGTCCGCCGGGCCCACCGGGCCC
GCCGGGCTTAACCGGTCCGGCCGGCGAGCCGGGCCGTGAAGGCAGCCCGGGCGCCG
ATGGCCCACCGGGCCGCGATGGCGCCGCGGGCGTGAAGGGTGATCGCGGTGAGACC
GGCGCCGTGGGCGCCCCGGGCGCGCCGGGTCCGCCGGGCGACCGCGGCGAGGCCGG
TGCGCAAGGTCCGATGGGCCCGAGCGGTCCGGCCGGTGCGCGTGGCATTCAAGGCC
CGCAAGGCCCACGCGGTGATAAAGGCGAAGCCGGTGAACCGGGCGAACGCGGCCTG
AAAGGCCATCGTGGTTTTACCGGTTTACAAGGTCTGCCGGGCCCGCCGGGCCCAAGT
GGTCCACCGGGCCCGCCGGGCCCACCGGGCCCACCGGGCCCACCGGGCCCGCCGGG
CTAA
]SEQ ID NO: 19: an amino acid sequence of V-HC3A
]HHHHHHADEQEEKAKVRTELIQELAQGLGGIEKKNFPTLGDEDLDHTYMTKLL
TYLQEREQAENSWRKRLLKGIQDHALDLVPRGSPGPPGPPGPPGPPGPPGFPGMKGHRG
FDGRNGEKGETGAPGLKGENGLPGENGAPGPMGPRGAPGERGSPGPKGDKGEPGPPGA
DGVPGKDGPRGPTGPIGPPGPAGQPGDKGEPGPPGPPGPPGPPGFPGMKGHRGFDGRNG
EKGETGAPGLKGENGLPGENGAPGPMGPRGAPGERGSPGPKGDKGEPGPPGADGVPGK
DGPRGPTGPIGPPGPAGQPGDKGEPGPPGPPGPPGPPGPPGPPG
]SEQ ID NO: 20: a nucleotide sequence of V-HC3A
CATCACCATCACCATCATGCGGATGAACAAGAAGAAAAAGCGAAAGTGCGC
ACCGAACTGATTCAAGAACTGGCGCAAGGCCTGGGCGGCATTGAAAAAAAAAACTT
TCCGACCCTGGGCGATGAAGATCTGGATCATACCTATATGACCAAACTGCTGACCTAT
CTGCAAGAACGCGAACAAGCGGAAAACAGCTGGCGCAAACGCCTGCTGAAAGGCAT
TCAAGATCATGCCCTGGATTTAGTGCCGCGCGGCAGCCCGGGTCCACCGGGTCCGCC
GGGCCCGCCGGGCCCACCGGGTCCGCCGGGCTTTCCGGGCATGAAGGGCCATCGCG
GTTTTGATGGCCGCAACGGCGAAAAAGGCGAAACGGGTGCCCCGGGCCTGAAAGGC
GAAAACGGTTTACCGGGCGAGAACGGCGCGCCGGGCCCGATGGGTCCGCGTGGTGC
GCCGGGCGAACGCGGCAGCCCGGGCCCAAAAGGTGATAAGGGTGAACCGGGTCCGC
CGGGCGCCGACGGTGTGCCGGGCAAAGATGGCCCGCGCGGCCCGACGGGCCCGATT
GGCCCGCCGGGCCCGGCGGGCCAACCGGGCGACAAAGGTGAACCGGGCCCGCCGG
GCCCGCCGGGCCCACCGGGTCCACCGGGTTTTCCGGGCATGAAGGGCCATCGCGGCT
TTGATGGTCGTAACGGCGAGAAGGGCGAAACCGGTGCGCCGGGCTTAAAAGGTGAA
AACGGCCTGCCGGGCGAGAACGGCGCGCCGGGTCCGATGGGCCCACGTGGCGCCCC
GGGCGAGCGCGGCAGTCCGGGCCCGAAGGGCGATAAAGGCGAACCGGGCCCGCCG
GGCGCGGATGGCGTGCCGGGCAAAGATGGCCCACGCGGTCCAACGGGTCCGATCGG
CCCGCCGGGCCCGGCGGGTCAGCCGGGCGATAAGGGTGAGCCGGGCCCGCCGGGCC
CGCCGGGCCCGCCGGGCCCGCCGGGCCCACCGGGCCCACCGGGTTAA
SEQ ID NO: 21:
LVPRGSP
]SEQ ID NO: 22:
LVPRGS
SEQ ID NO: 23:
GPPGPPGPPGPPGPPGPPGPPGPPGPPGPP
SEQ ID NO: 24:
GPPGPPGPPGPPGPP
SEQ ID NO: 25:
GPPGPPGPPGPP
SEQ ID NO: 26:
GPPGPPGPPGPPGPPGPP
SEQ ID NO: 27 (an amino acid sequence of KD2):
GPPGPPGPKGDPGPPGPPGPKGDPGPPGPP
SEQ ID NO: 28 (an amino acid sequence of KD3):
GPPGPKGDPGPPGPKGDPGPPGPKGDPGPP
SEQ ID NO: 29 (an amino acid sequence of V-KD3BP10):
]HHHHHHHHGGGGSADEQEEKAKVRTELIQELAQGLGGIEKKNFPTLGDEDLDH
TYMTKLLTYLQEREQAENSWRKRLLKGIQDHALDLVPRGSPGPPGPKGDPGPPGPKGDP
GPPGPKGDPGPPGFPGERGVQGPPGPAGPRGANGAPGNDGAKGDAGAPGAPGSQGAPG
LQGMPGERGAAGLPGPKGDRGDAGPKGADGSPGKDGVRGLTGPIGPPGPAGAPGDKGE
SGPSGPPGPPGPPGPPGPPGPPGPPGPPGPPGPPG
SEQ ID NO: 30 (an amino acid sequence of V-P10BKD3):
HHHHHHHHGGGGSADEQEEKAKVRTELIQELAQGLGGIEKKNFPTLGDEDLDH
TYMTKLLTYLQEREQAENSWRKRLLKGIQDHALDLVPRGSPGPPGPPGPPGPPGPPGPPG
PPGPPGPPGPPGFPGERGVQGPPGPAGPRGANGAPGNDGAKGDAGAPGAPGSQGAPGL
QGMPGERGAAGLPGPKGDRGDAGPKGADGSPGKDGVRGLTGPIGPPGPAGAPGDKGES
GPSGPPGPKGDPGPPGPKGDPGPPGPKGDPGPPG
]SEQ ID NO: 31 (an amino acid sequence of V-P10BKD2):
HHHHHHHHGGGGSADEQEEKAKVRTELIQELAQGLGGIEKKNFPTLGDEDLDH
TYMTKLLTYLQEREQAENSWRKRLLKGIQDHALDLVPRGSPGPPGPPGPPGPPGPPGPPG
PPGPPGPPGPPGFPGERGVQGPPGPAGPRGANGAPGNDGAKGDAGAPGAPGSQGAPGL
QGMPGERGAAGLPGPKGDRGDAGPKGADGSPGKDGVRGLTGPIGPPGPAGAPGDKGES
GPSGPPGPPGPKGDPGPPGPPGPKGDPGPPGPPG
SEQ ID NO: 32 (an amino acid sequence of V-P10BP10):
HHHHHHHHGGGGSADEQEEKAKVRTELIQELAQGLGGIEKKNFPTLGDEDLDH
TYMTKLLTYLQEREQAENSWRKRLLKGIQDHALDLVPRGSPGPPGPPGPKGDPGPPGPP
GPKGDPGPPGPPGFPGERGVQGPPGPAGPRGANGAPGNDGAKGDAGAPGAPGSQGAPG
LQGMPGERGAAGLPGPKGDRGDAGPKGADGSPGKDGVRGLTGPIGPPGPAGAPGDKGE
SGPSGPPGPPGPKGDPGPPGPPGPKGDPGPPGPPG
SEQ ID NO: 33 (an amino acid sequence of V-KD2BKD2):
HHHHHHHHGGGGSADEQEEKAKVRTELIQELAQGLGGIEKKNFPTLGDEDLDH
TYMTKLLTYLQEREQAENSWRKRLLKGIQDHALDLVPRGSPGPPGPPGPPGPPGPPGPPG
PPGPPGPPGPPGFPGERGVQGPPGPAGPRGANGAPGNDGAKGDAGAPGAPGSQGAPGL
QGMPGERGAAGLPGPKGDRGDAGPKGADGSPGKDGVRGLTGPIGPPGPAGAPGDKGES
GPSGPPGPPGPPGPPGPPGPPGPPGPPGPPGPPG

Although the present disclosure has been disclosed in preferred examples, but they are not intended to limit the present disclosure. Anyone skilled in the art can make various changes and modifications without departing from the spirit and scope of the present disclosure. Therefore, the scope of protection of the present disclosure should be defined by the claims.

What is claimed is:

1. An amino acid sequence encoding a collagen domain, comprising:
(1) an amino acid sequence set forth in any one of SEQ ID NO:1-7; or
(2) an amino acid sequence obtained by combining any two sequences set forth in SEQ ID NO: 1-3; or
(3) an amino acid sequence obtained by repeating a sequence set forth in any one of SEQ ID NO: 1-7 for 2-3 times.

2. A single protein chain for expressing collagen, comprising the amino acid sequence encoding a collagen domain according to claim 1.

3. The single protein chain according to claim 2, wherein a structure of the single protein chain from an N-terminus to a C-terminus comprises: a folding domain, {a repeating sequence module, a collagen domain}m, and a repeating sequence module, wherein m is an integer and m≥1.

4. The single protein chain according to claim 3, wherein a front terminus of the folding domain further has a histidine tag.

5. The single protein chain according to claim 3, wherein the folding domain assists collagen folding to form a triple helix structure; and
the folding domain is a V-domain or a coiled-coil domain; the folding domain is a V-domain, with an amino acid sequence set forth in SEQ ID NO:13; and the folding domain is a coiled-coil domain, with an amino acid sequence set forth in SEQ ID NO: 14.

6. The single protein chain according to claim 3, wherein a plurality of the repeating sequence modules are arranged and located at both termini of a collagen domain or both termini of a plurality of collagen domains; and
sequences of the repeating sequence modules are identical or different; the repeating sequence modules employ (GPP)ₙ; and a value of n in (GPP)ₙ satisfies 5<n≤30.

7. The single protein chain according to claim 2, wherein a structure of the single protein chain for expressing collagen is as follows: a first repeating sequence module-a collagen domain-a second repeating sequence module;
at least one of the first repeating sequence module and the second repeating sequence module has an amino acid sequence set forth in SEQ ID NO:27 or SEQ ID NO:28, and the other module has an amino acid sequence set forth in SEQ ID NO:23; and
or both the first repeating sequence module and the second repeating sequence module have the amino acid sequence set forth in SEQ ID NO:23.

8. A nucleotide sequence encoding the collagen domain according to claim 1.

9. A gene encoding the single protein chain according to claim 7.

10. A collagen, wherein three single protein chains according to claim 2 intertwine around a common central axis to form a triple helix structure.

11. A collagen, comprising the single protein chain according to claim 7.

12. Collagen fibers formed by high-polymerization-induced self-assembly of the collagen according to claim 10.

13. A product containing collagen with a triple helix structure, wherein the collagen with a triple helix structure has the collagen domain of an amino acid sequence according to claim 1.

14. The product containing collagen with a triple helix structure according to claim 13, wherein the product is a product in the fields of beauty, chemical engineering, food health, medical treatment/biomedicine, cosmetics, and feed, beauty cosmetics (a facial mask, an essence, a cream, or the like), an artificial collagen casing, a nutritional health product (a collagen powder, an oral liquid), a medical dressing, a hemostatic material, an artificial bone scaffold, an injectable filler, an artificial blood vessel, an eye drop, or a sustained-release drug carrier.

15. A collagen product, comprising the single protein chain according to claim 7.

16. The collagen product according to claim 15, further comprising one or more of a vitamin, a mineral, hyaluronic acid, a natural polysaccharide, an essential oil, a polyphenolic substance, and a natural plant extract.

17. A method for expressing collagen with a triple helix structure, wherein the method is used to construct collagen with a specific collagen domain; and an amino acid sequence encoding the collagen domain comprises:
(1) an amino acid sequence set forth in any one of SEQ ID NO: 1-7; or
(2) an amino acid sequence obtained by combining any two sequences set forth in SEQ ID NO: 1-3; or
(3) an amino acid sequence obtained by repeating a sequence set forth in any one of SEQ ID NO: 1-7 for 2-3 times.

18. A method for improving stability, solubility, or yield of collagen, comprising: linking a first repeating sequence module to an N-terminus of a collagen domain, and linking a second repeating sequence module to a C-terminus to construct a single collagen chain with a structure of first repeating sequence module-collagen domain-second repeating sequence module, and expressing through a host cell; at least one of the first repeating sequence module and the second repeating sequence module has an amino acid sequence set forth in SEQ ID NO:27 or SEQ ID NO: 28;
an amino acid sequence of the collagen domain comprises:
(1) an amino acid sequence set forth in any one of SEQ ID NO: 1-7; or
(2) an amino acid sequence obtained by combining any two sequences set forth in SEQ ID NO: 1-3; or
(3) an amino acid sequence obtained by repeating a sequence set forth in any one of SEQ ID NO: 1-7 for 2-3 times.
